(19) United States
(12) Patent Application Publication
(10) Pub. No.: US 2022/0202397 A1
(43) Pub. Date: Jun. 30, 2022

(54) SYSTEMS AND METHODS FOR LIQUID FLOODING OF LUNG TO ENHANCE ENDOBRONCHIAL ENERGY TRANSFER FOR USE IN IMAGING, DIAGNOSIS AND/OR TREATMENT

(71) Applicant: Ethicon, Inc., Somerville, NJ (US)

(72) Inventors: Megan Greenwood, Houston, TX (US); Matthew Kuhn, Houston, TX (US); Joanna Nathan, Houston, TX (US)

(21) Appl. No.: 17/522,317

(22) Filed: Nov. 9, 2021

Related U.S. Application Data
(60) Provisional application No. 63/132,512, filed on Dec. 31, 2020.

(51) Int. Cl. A61B 8/08; A61B 8/12; A61B 10/04; A61B 34/30; A61B 34/00; A61B 34/20; A61N 7/02; A61B 8/00; A61K 49/22

(52) U.S. Cl. CPC A61B 8/481; A61N 2007/0043; A61B 8/085; A61B 10/04; A61B 34/30; A61B 34/25; A61B 34/20; A61N 7/022; A61B 8/0858; A61B 8/546; A61K 49/22; A61B 2034/301; A61B 2034/2063; A61B 2034/2051; A61B 8/12

(57) ABSTRACT

An improved system and method of endobronchial imaging of lung nodules comprises the introduction of a perfluorocarbon (PFC) liquid into pulmonary passages of the lungs, the introduction of which enables better coupling between an endobronchial ultrasonic imaging system and a target tissue site within the pulmonary passages of the lungs, the improved coupling between the ultrasonic imaging system and a target tissue site being imparted by the removal (at least in part) the air interface present between the ultrasonic imaging system and the surface of the target tissue site. Furthermore, the unique properties of perfluorocarbon liquids (for example, the properties of superb biocompatibility, high affinity for dissolving oxygen, and extremely low surface tension) further position these substances to be particularly well-suited for this application.

### CROSS-REFERENCE TO RELATED APPLICATIONS

This application claims priority to and the benefit of U.S. Patent Application No. 63/132,512 filed Dec. 31, 2020, which is hereby incorporated by reference in their entirety.

### BACKGROUND OF THE DISCLOSURE

### 1. Field of the Disclosure

The present disclosure relates to imaging of the lungs, and more particularly to an improved system and method of endobronchial imaging of lung nodules which comprises the introduction of an oxygen carrying liquid into pulmonary passages of the lungs, the introduction of which enables better coupling between an endobronchial ultrasonic imaging system and a target tissue site within the pulmonary passages of the lungs, the improved coupling between the ultrasonic imaging system and a target tissue site being imparted by the removal (at least in part) the air interface present between the ultrasonic imaging system and the surface of the target tissue site.

### 2. Discussion of the Related Art

Ultrasound imaging utilizes sound waves to create an image. More specifically, an ultrasound machine transmits high-frequency sound pulses, for example 1 to 20 megahertz, into a body utilizing a probe. The sound waves travel into the body and hit a boundary between tissues, for example, between fluid and soil tissue and soft tissue and bone. The reflected waves are picked up by the probe and relayed to the machine. An ultrasound image is produced based on the reflection of the sound waves off of the body structures. The strength or amplitude of the sound signal and the time it takes for the wave to travel through the body provide the information necessary to produce an image.

Ultrasound imaging may be utilized in the diagnosis of a wide range of diseases and conditions. However, it cannot be utilized to image all areas and organs of the body. Ultrasound cannot be utilized to image bones because they are too dense to penetrate. Additionally, the intestinal track and normal lung tissue are not easily identified with ultrasound because air or other gas interferes with the production of ultrasound images. Accordingly, there exists a need for an improved system and method of endobronchial imaging of lung nodules.

### SUMMARY

An improved system and method of endobronchial imaging of lung nodules comprises the introduction of an oxygen carrying liquid into pulmonary passages of the lungs, the introduction of which enables better coupling between an endobronchial ultrasonic imaging system and a target tissue site within the pulmonary passages of the lungs, the improved coupling between the ultrasonic imaging system and a target tissue site being imparted by the removal (at least in part) the air interface present between the ultrasonic imaging system and the surface of the target tissue site. As an illustrative example, the introduced liquid may preferably comprise, in part or in whole, one or more perfluorocarbon liquids (PFCs), however, the introduced liquid may also comprise saline, water, a pharmaceutical solution, or any other biocompatible liquid. Furthermore, the unique properties of perfluorocarbon liquids (for example, the properties of superb biocompatibility, high affinity for dissolving oxygen, and extremely low surface tension) further position these substances to be particularly well-suited for this application.

Additional use cases are listed herein. These include enhanced imaging techniques such as optical, electrical bioimpedance, thermal, photoacoustic, and other combination modalities. These imaging techniques can be used to accurately locate the nodule for biopsy or to characterize the tissue for diagnosis. Additionally, fluid collection may be used as a means of diagnosis. Treatment options may also be enhanced by lung flooding and include high intensity focused ultrasound (HIFU), electroporation, radiofrequency ablation (RFA), cryoablation, and microwave ablation (MWA), and targeted drug delivery.

### BRIEF DESCRIPTION OF THE DRAWINGS

The foregoing and other features and advantages of the disclosure will be apparent from the following, more particular description of embodiments of the disclosure, as illustrated in the accompanying drawings.

FIG. 1A is a diagrammatic representation of an exemplary operating environment for implementing a biopsy in accordance with the present disclosure;

FIG. 1B is a diagrammatic representation of a luminal network navigated for biopsy in accordance with the present disclosure;

FIG. 1C is a diagrammatic representation of a luminal network navigated for biopsy filled with a liquid medium in accordance with the present disclosure;

FIG. 1D is a diagrammatic representation of a luminal network navigated for biopsy filled with a liquid medium constrained to a localized area in accordance with the present disclosure;

FIG. 1E-1F are diagrammatic representations of a dual balloon occlusion mechanism for constraining the liquid medium to a localized area.

FIG. 1G-1I are diagrammatic representations of non-balloon embodiments of the occlusion mechanism for constraining the liquid medium to a localized area.

FIG. 2 is a diagrammatic representation of an exemplary robotic arm for guiding an instrument to sample locations within a luminal network in accordance with the present disclosure;

FIG. 3 is a diagrammatic representation of a distal end of an endoscope in accordance with the present disclosure; and

FIG. 4 is a flow chart of the overall process in accordance with the present disclosure.

FIG. 5 is a flow chart of the process for filling the lung with liquid medium.

FIGS. 6A-6D is a representation of ultrasound images able to be obtained in different lung conditions.

FIGS. 7A-7B are representations of x-ray and CT images of a small nodule located in the periphery of the lung.

FIGS. 8A-8B are diagrammatic representations of the unsuccessful endobronchial biopsy of a small nodule located in the periphery of the lung.

FIGS. 9A-9B are representations of ultrasound images of a small nodule targeted for endobronchial biopsy before and after localized filling of the lung with liquid medium.

### DETAILED DESCRIPTION

Perfluorocarbon liquids (PFCs) are clear, colorless, odorless, nonflammable, chemically inert liquids that are unique in their high affinity for respiratory gases. PFCs have an oxygen-carrying capacity up to fifty (50) times more than that of human plasma. PFCs are derived from common organic compounds by the replacement of all carbon-bound hydrogen atoms with fluorine atoms. They are denser than water and soft tissue, have low surface tension, and low viscosity. PFCs have the lowest sound speeds of all liquids.

Like other highly inert carbon fluorine materials, PFCs are extremely nontoxic and biocompatible. Numerous studies have shown that mammals can breathe oxygenated perfluorocarbon liquids for long periods (>3 hours) and return to gas breathing without untoward long-term effects. Additional studies have shown that no adverse morphological, biochemical, or histological effects are seen after perfluorocarbon ventilation. PFCs have also shown considerable therapeutic potential via intravenous delivery in supporting intravascular gas transport.

Oxygen is physically dissolved in (PFCs) and 90 percent-100 percent is released by simple diffusion in response to concentration gradients of respiratory gases according to Henry's Law. PFCs dramatically enhance oxygen delivery from red blood cells to tissues, with up to a 50-fold increase in oxygen diffusion rates in some studies thereby making the PFCs an oxygen supercharger. The high oxygen-carrying capacity makes PFCs ideal candidates for supporting respiratory gas exchange via infusion directly into the lungs, which may help mitigate the risk of ischemic or hypoxic events during endobronchial imaging within a fluid medium introduced into the lung.

Lung cancer is by far the leading cause of cancer death among both men and women. According to the American Cancer Society: each year, more people die of lung cancer than of colon, breast, and prostate cancers combined. The American Lung Association notes that the 5-year survival rate for lung cancer is 56 percent when the disease is still localized. However, only 16% of lung cancer cases are diagnosed at an early stage. For distant tumors, the five-year survival rate is only 5 percent. The National Lung Screening Trial (NLST), published in 2011, demonstrated that routine screening resulted in a 20% mortality reduction in high risk patients, proving that the best way to positively impact lung cancer survival rates is early diagnosis and treatment.

Diagnosis of lung cancer is commonly achieved via endobronchial biopsy technologies, which navigate through the airways of the lung to an identified nodule. The effectiveness of endobronchial biopsy technologies is limited for the biopsy of small- to intermediate-sized (4 mm to 12 mm in diameter) nodules. Even with CT-guidance, the small size of these nodules presents a difficult challenge for accurate targeting with an endobronchial biopsy needle, and many of these small nodules are deemed to be too difficult to biopsy. Alternatively, percutaneous biopsy can also be used as a method of collecting lung tissue for the diagnosis of lung cancer, however, percutaneous biopsy of small- to intermediate-sized nodules remains a challenge and requires a high degree of technical skill, requiring longer procedure times, and often the need for multiple needle adjustments that increase the risk of complications. Currently, real-time confirmation of nodule location, which would greatly reduce the difficulty of endobronchial biopsy of lung nodules, cannot be readily achieved with ultrasound due to the widespread presence of air within the lung. The air-filled pockets within the lung significantly block the transmission of ultrasound waves through tissue, therefore making the use of ultrasound imaging devices impossible. As a result of the inability to image lung nodules in real-time, only 60% of small to intermediate sized nodules sampled via endobronchial needle biopsy are successfully diagnosed.

Although lung cancer can occur anywhere in the lungs, about three-quarters of primary lung cancers occur in and/or on the bronchial walls within the first three bronchial generations, i.e., near or proximal to the hilus, the region where the airways and major vessels enter and leave each lung. Many tumors occur near the carina, at the junction of the right and left bronchi with the trachea, presumedly due to increased deposition of inhaled carcinogens. Squamous cell carcinoma tumors, one of the most common histological type, making up 30-40% of lung tumors, arise inside the surface layer of the bronchial wall and then invade the wall and adjacent structures.

Because of its relative safety and utility as a non-invasive means of imaging tissues within the body, ultrasound is frequently used to image tumors. However, the presence of air in the lung has precluded ultrasound from implementation in the application of non-invasive imaging of tumors within the lung. The present disclosure solves this problem, in the preferred embodiment, by an unconventional use of “breathable liquids” (e.g., perfluorocarbon liquids) and an ultrasonic imaging system. As used herein, the phrase “breathable liquids” refers to liquids which have the ability to deliver oxygen into, and to remove carbon dioxide from, the pulmonary system (i.e., the lungs) of patients. Examples of breathable liquids include, but are not limited to, saline, silicone, and vegetable oils, perfluorochemicals, and the like. One of the presently preferred breathable liquids is perfluorocarbon liquids.

Without a means for non-irradiative, real-time confirmation of nodule location relative to an endobronchial biopsy device, diagnosis of lung cancer will remain a significant challenge with current endobronchial needle biopsy techniques. Clinicians need a tool that can provide real-time ultrasonic imaging of lung nodules targeted for endobronchial biopsy to help verify proper positioning and alignment of an endobronchial biopsy device relative to a nodule so that an accurate needle biopsy and confident diagnosis of cancer can be achieved.

An endobronchial biopsy device may comprise a surgical robotic system with one or more robotic arms for positioning and guiding movement of endoscope through the luminal network of the patient and, in some cases, actuating a collection device (e.g., a biopsy needle, brush, forceps, or the like). A control module can be communicatively coupled to the surgical robotic system for receiving position data and/or providing control signals from a user.

The endoscope may be a tubular and flexible surgical instrument that is inserted into the anatomy of a patient to capture images of the anatomy (e.g., body tissue, target tissue site) and provide a working channel for insertion of other medical instruments to a target tissue site. The endoscope can include one or more location sensors at its distal end. The one or more location sensors may comprise imaging elements such as one or more ultrasound transducers, optical cameras, and/or EM sensors. The imaging elements move along with the tip of the endoscope such that movement of the tip of the endoscope results in corresponding changes to the field of view of the images captured by the imaging elements.

In accordance with exemplary embodiments of the present disclosure, the distal end of the endoscope can be provided with one or more ultrasound transducers (e.g., radial-scanning, or linear-scanning ultrasound transducers) configured to produce images of the anatomy (e.g., body tissue). The images of the anatomy produced from the ultrasound transducers may be used to identify position and/or orientation of the distal end of the endoscope. In some embodiments, one or more models of the anatomy of the patient may be used together with the images of the anatomy to identify position and/or orientation of the distal end of the endoscope. As an example, a preoperative procedure can be performed to take CT scans of a patient's lungs, and a computing system can use data from these scans to build a 3D model of the lungs of the patient. Such a model can provide 3D information about the structure and connectivity of the lung luminal network, including the topography and/or diameters of patient airways in some examples. Some CT scans are performed at breath-hold so that the patient's airways are expanded to their full diameter. Then, this model of the luminal network may be used in conjunction with the images from the one or more of imaging elements at the distal end of the endoscope to determine position and/or orientation of the distal end.

The endobronchial biopsy device may include one or more user interface screens, such as electronic monitors (e.g., LCD displays, LED displays, touch-sensitive displays), virtual reality viewing devices, e.g., goggles or glasses, and/or other display devices. In some embodiments, one of the displays may display a virtual representation of the biopsy pattern or one or more sample locations within the biopsy pattern. In some embodiments, one of the displays can display a 3D model of the patient's luminal network and virtual biopsy information (e.g., a virtual representation of the biopsy pattern in the target tissue site or a virtual representation of paths of the end of the endoscope toward sample locations of the biopsy pattern within the model based on EM sensor position) while the other of the displays can display image information received from an ultrasonic imaging device at the end of the endoscope.

A user may compare the virtualized display of the endoscope within a 3D model of a patient's anatomy to actual images captured by an ultrasonic imaging system to help mentally orient and confirm that the endoscope is in the correct—or approximately correct—location within the patient. The display may simultaneously display the 3D models of the endoscope and the anatomy the around distal end of the endoscope. Further, the display may overlay images obtained via an ultrasonic imaging system with the 3D model and CT scans.

The distal end of the endoscope may include one or more working channels through which surgical instruments, such as biopsy needles, cytology brushes, and forceps, may be inserted along the endoscope shaft, allowing access to the area near the endoscope tip. Fluids, such as perfluorocarbon liquids, may additionally be delivered via the working channel of the endoscope of the robotic system to enable intracavity ultrasonic imaging of nodules targeted for biopsy. The endoscope system may be designed such that the surgical instruments may be deployed through the working channels of the endoscope concurrently with or separately from the deployment of the ultrasound probe.

A biopsy pattern data may comprise one or more sample locations at which biopsy samples are to be collected. The biopsy patterns may be selected or modified by a user. In embodiments in which the medical instrument includes an ultrasound transducer at the distal end of the instrument, the user may modify the biopsy pattern to more accurately reflect the real-time position of a nodule targeted for biopsy.

In some implementations, the robotic endobronchial biopsy system may implement object recognition techniques, by which the system can detect objects present in the field of view of the ultrasound image data, such as branch openings, lesions, nodules, or particles. Using object recognition, the robotic endobronchial biopsy system can output object data indicating information about what objects were identified, as well as positions, orientations, and/or sizes of the recognized objects. The robotic endobronchial biopsy system may then adjust parameters governing the position of the distal end of the endoscope and/or the planned biopsy trajectory to optimize accurate biopsy sample retrieval.

An exemplary method of real-time endobronchial ultrasonic imaging of nodules within the lung involves the steps of:
1. Temporarily filling with a liquid medium preselected pulmonary air passages adjoining pulmonary tissues containing indeterminate nodules targeted for biopsy.
   a. By “pulmonary air passages” it is meant the pulmonary channels, spaces, or volumes in the trachea, left and right bronchi, bronchioles, and alveoli of the lungs that are normally occupied by air.
      i. In the practice of the disclosure, only the pulmonary air passages in contact with or near a patient's tumor site(s) are typically filled with the liquid medium, and gaseous ventilation of the remaining pulmonary air passages is maintained.
   b. Depending on the location of the lung nodule, as determined by available diagnostic and/or imaging methods, the fluid-filled pulmonary air passages may be localized in a lung, lobe, or lung segment, and/or at least a portion of the bronchial tree may be selected for localized filling with the liquid medium.
   c. Localized filling of the pulmonary air passages in such a preselected manner can be effected by means one or more infusion catheters, a bronchoscope, and/or an endobronchial biopsy device.
      i. One or more balloons may be inflated in the air passageways to isolate the preselected regions of air. Alternately, other occlusion devices such as flaps, valves, expandable devices, etc., may be used instead of balloons to isolate the preselected regions.
         1. A proximal balloon may be inflated prior to the localized filling to prevent liquid egress proximal to the balloon location in the bronchial tree.
         2. Optionally and additionally, a distal balloon may be inflated prior to the localized filling to prevent liquid egress distal to the balloon; thereby limiting the fluid infiltration to the bronchial tree region within the proximal and distal balloons.
   d. Several imaging modalities—such as ultrasound, fluoroscopy, optics, or any combination thereof—may be used to monitor the filling of the pulmonary air passages.
   e. The liquid medium introduced into the lungs to enable endobronchial ultrasonic imaging may also contain a therapeutic agent such as an anti-cancer drug (e.g., adriamycin), toxin, antibody-linked radionuclide, etc.
   f. A preferred liquid medium for this treatment is a perfluorocarbon liquid of the general type used for lung ventilation.
   g. In order to serve as a suitable acoustical propagating medium, the perfluorocarbon liquid may preferably have the following physical, thermal, and acoustical properties:
      i. Viscosity less than about 5 CP at 25° C.
      ii. Density less than about 2.0 g/cm3 at 25° C.
      iii. Acoustic impedance between about 0.8 to about 1.6 MegaRayls at 37° C.
      iv. Acoustic attenuation less than about 1.2 dB/cm (±20%) at 1.0 MHz, 45° C.
      v. Acoustic intensity of about 3 W/cm2
   h. The perfluorocarbon liquid is preferably characterized by an oxygen solubility greater than about 40 ml/100 ml
2. Introducing an ultrasonic imaging device into the lungs.
   a. The ultrasound imaging device may comprise an intracavity transducer disposed within the liquid-filled pulmonary air passages, or the transducer may be disposed exogenous to the liquid-filled pulmonary air passages.
   b. The ultrasound imaging device may comprise a transducer integrated into an end-effector of robotic endobronchial biopsy device such as the Auris Monarch.
   c. The ultrasound may be transmitted through an intercostal space of the patient, or it may be transmitted from an exposed surface of the lung into the volume of same during an intra-operative application involving an “acoustic window” into the lung created by surgical means.
   d. The ultrasound transducer can be applied to the pulmonary pleura or lung surface overlying the fluid-filled passages, following surgical displacement of ribs or other interfering tissues.
3. Causing the ultrasonic imaging device to transmit ultrasonic energy through the tissue of the lungs for obtaining real-time positioning of one of more nodules targeted for biopsy.
   a. In cases where the preselected liquid-filled pulmonary air spaces are localized in the bronchial tree, the ultrasound from an intracavitary transducer preferably has a frequency in the range of from about 250 KHz to about 3 MHz, and most preferably from about 500 KHz to about 2 MHz.
   b. For peripheral lung treatments (i.e., in the membranous airways and alveoli of the lung), where the sound waves must necessarily traverse many more liquid-tissue interfaces, a lower ultrasound frequency in the range of from about 250 KHz to about 1.5 MHz may be preferred when perfluorocarbon liquids serve as the liquid medium.
   c. Ultrasound frequencies in the range of from about 250 KHz to about 1.5 MHz may be preferred when the transducer is positioned exogenous to the lung.
   d. The desired frequency ranges are established on the basis of the depth of imaging sought. Lower frequencies are attenuated less and, therefore, are employed where imaging deeper nodules is preferred. Conversely, higher frequencies are more readily absorbed, and thus are more appropriate for more superficial imaging.
   e. An ultrasound transducer may broadcast at more than one frequency to optimize imaging. The changes in frequency in this case may be done by rapid incremental changes in frequency over a specified bandwidth using frequency modulation (FM) methods, or they may be done with serial changes over time whereby sound (in FM mode or not) is generated in predetermined frequency ranges for desired periods and then changed to other frequencies for periods of time.
   f. Optimal imaging may comprise the use of multiple ultrasonic transducers (focused, diverging, or unfocused) operating in tandem at similar or different frequencies (in FM mode or not).
4. Leveraging data from the ultrasonic imaging system may be used to inform endobronchial biopsy device positioning relative to a nodule targeted for biopsy and enable more accurate biopsy. Optionally and additionally, real-time ultrasound imaging may be used to adjust or otherwise modify biopsy pattern data, visualize biopsy device movement and positioning relative to tissue(s) of interest, or any combination thereof.
5. After successful biopsy/biopsies, the liquid medium is removed from the pulmonary air passages of the patient.
   a. Removal of the liquid medium from the pulmonary air passages can be effected by means one or more infusion catheters, a bronchoscope, and/or an endobronchial biopsy device.
   b. Removal may comprise allowing the liquid medium to evaporate passively.
   c. Removal may comprise repositioning the patient to allow for gradual drainage of the liquid medium from the pulmonary air passages.

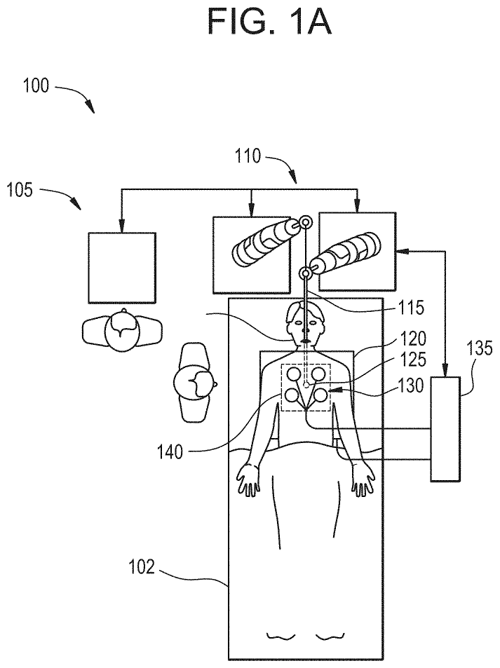

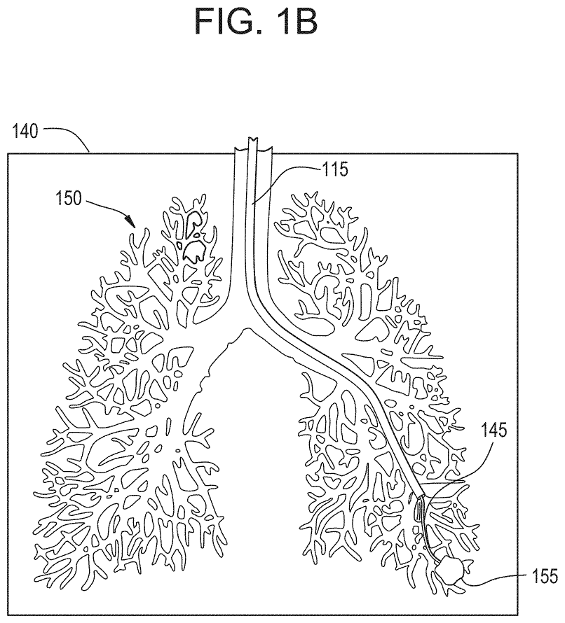

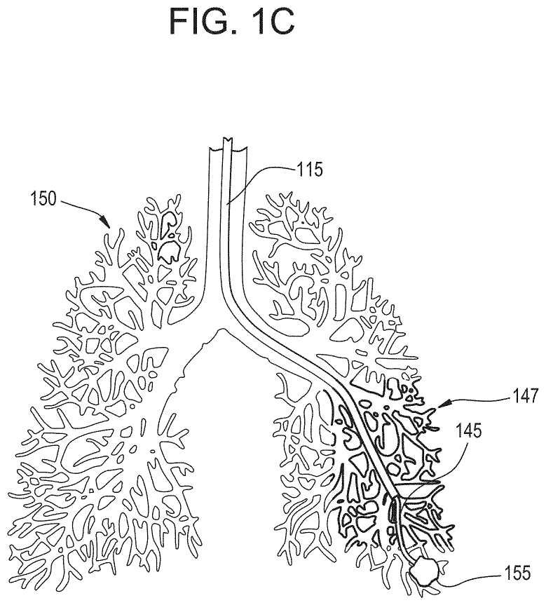

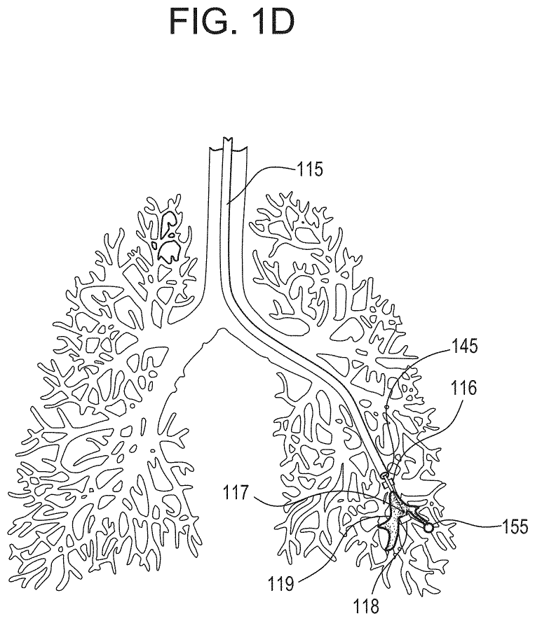

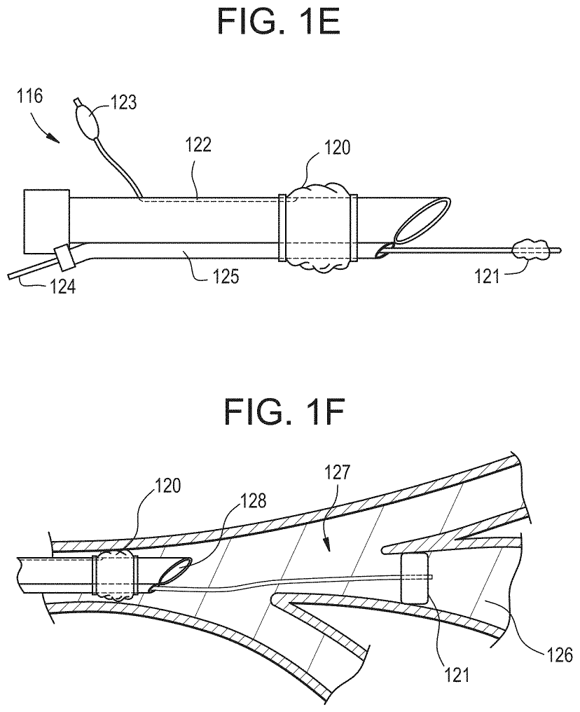

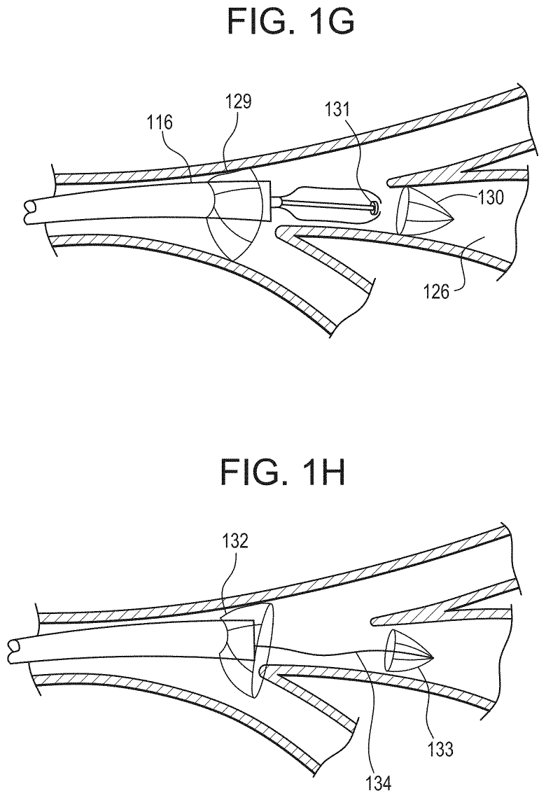

Referring now to FIG. 1A, there is illustrated an exemplary operating environment 100 implementing one or more aspects of the disclosed biopsy systems and techniques. The operating environment 100 includes patient 101, a platform 102 supporting the patient 101, a surgical robotic system 110 guiding movement of endoscope 115, command center 105 for controlling operations of the surgical robotic system 110, electromagnetic (EM) controller 135, EM field generator 120, and EM sensors 125, 130. FIG. 1A also illustrates an outline of a region of a luminal network 140 within the patient 101.

The surgical robotic system 110 may include one or more robotic arms for positioning and guiding movement of endoscope 115 through the luminal network 140 of the patient 101 and, in some cases, actuating a collection device (e.g., a biopsy needle, brush, forceps, or the like). Command center 105 may be communicatively coupled to the surgical robotic system 110 for receiving position data and/or pro viding control signals from a user. As used herein, “communicatively coupled” refers to any wired and/or wireless data transfer mediums, including but not limited to a wire less wide area network (WWAN) (e.g., one or more cellular networks), a wireless local area network (WLAN) (e.g., configured for one or more standards, such as the IEEE 802.11 (Wi-Fi)), Bluetooth, data transfer cables, and/or the like.

The endoscope 115 may be a tubular and flexible surgical instrument that is inserted into the anatomy of a patient to capture images of the anatomy (e.g., body tissue, target tissue site) and provide a working channel for insertion of other medical instruments to a target tissue site. In some implementations, the endoscope 115 may be a bronchoscope. The endoscope 115 may include one or more location sensors at its distal end. The one or more location sensors may comprise imaging devices (e.g., cameras or other types of optical sensors), ultrasound transducers, X-ray devices (e.g., X-ray image intensifiers, X-ray imaging devices, and fluoroscopy devices) and/or EM sensors. The imaging devices may include one or more optical components such as an optical fiber, fiber array, photosensitive substrate, and/or lens(es). The optical components move along with the tip of the endoscope 115 such that movement of the tip of the endoscope 115 results in corresponding changes to the field of view of the images captured by the imaging devices. The distal end of the endoscope 115 may be provided with one or more ultrasound transducers (e.g., radial-scanning, or linear-scanning ultrasound transducers) or X-ray devices configured to produce images of the anatomy (e.g., body tissue). The images of the anatomy produced from the imaging devices, the ultrasound transducers, and/or the X-ray devices may be used to identify position and/or orientation of the distal end of the endoscope 115. In some embodiments, one or more models of the anatomy of the patient may be used together with the images of the anatomy to identify position and/or orientation of the distal end of the endoscope 115. As an example, a preoperative procedure may be performed to take CT scans of a patient's lungs, and a computing system may use data from these scans to build a 3D model of the lungs of the patient. Such a model may provide 3D information about the structure and connectivity of the lung luminal network, including the topography and/or diameters of patient airways in some examples. Some CT scans are performed at breath-hold so that the patient's airways are expanded to their full diameter. Then, this model of the luminal network may be used in conjunction with the images from the one or more location sensors at the distal end of the endoscope 115 to determine position and/or orientation of the distal end.

In addition, the distal end of the endoscope 115 may be provided with one or more EM sensors for tracking the position of the distal end within an EM field generated around the luminal network 140.

EM controller 135 may control EM field generator 120 to produce a varying EM field. The EM field may be time-varying and/or spatially varying, depending upon the embodiment. The EM field generator 120 may be an EM field generating board in some embodiments. Some embodiments of the disclosed biopsy guidance systems may use an EM field generator board positioned between the patient and the platform 102 supporting the patient, and the EM field generator board may incorporate a thin barrier that minimizes any tracking distortions caused by conductive or magnetic materials located below it. In other embodiments, an EM field generator board may be mounted on a robotic arm, for example similar to those shown in surgical robotic system 110, which may offer flexible setup options around the patient.

An EM spatial measurement system incorporated into the command center 105, surgical robotic system 110, and/or EM controller 135 may determine the location of objects within the EM field that are embedded or provided with EM sensor coils, for example EM sensors 125, 130. When an EM sensor is placed inside a controlled, varying EM field as described herein, voltages are induced in the sensor coils. These induced voltages may be used by the EM spatial measurement system to calculate the position and/or orientation of the EM sensor and thus the object having the EM sensor. As the magnetic fields are of a low field strength and may safely pass through human tissue, location measurement of an object is possible without the line-of-sight constraints of an optical spatial measurement system.

EM sensor 125 may be coupled to a distal end of the endoscope 115 in order to track its location within the EM field. The EM field is stationary relative to the EM field generator, and a coordinate frame of a 3D model of the luminal network may be mapped to a coordinate frame of the EM field.

FIG. 1B illustrates an example luminal network 140 that may be navigated for biopsy in the operating environment 100 of FIG. 1A. The luminal network 140 includes the branched structure of the airways 150 of the patient and a nodule 155 (or lesion) that may be accessed as described herein for biopsy. As illustrated, the nodule 155 is located at the periphery of the airways 150. The endoscope 115 has a first diameter and thus its distal end is not able to be positioned through the smaller-diameter airways around the nodule 155. Accordingly, a steerable catheter 145 extends from the working channel of the endoscope 115 the remaining distance to the nodule 155. The steerable catheter 145 may have a lumen through which instruments, for example biopsy needles, cytology brushes, and/or tissue sampling forceps, may be passed to the target tissue site of nodule 155. In such implementations, both the distal end of the endoscope 115 and the distal end of the steerable catheter 145 may be provided with EM sensors for tracking their position within the airways 150. In other embodiments, the overall diameter of the endoscope 115 may be small enough to reach the periphery without the steerable catheter 145, or may be small enough to get close to the periphery (e.g., within 2.5-3 cm) to deploy medical instruments through a non-steerable catheter (not illustrated). The medical instruments deployed through the endoscope 115 may be equipped with EM sensors. FIG. 1C illustrates the PFC liquid delivered into the pulmonary passages at the target biopsy site 147.

FIG. 1D illustrates the endoscope 115 with a sheath or steerable catheter 145, proximal occlusion device 116, an imaging or biopsy tool 117 and a distal balloon 118 proximate nodule 155. The occlusion devices are used to limit the extent of lung flooding to a specific area of interest in the brachial tree. FIG. 1E-1G illustrate possible configurations of the occlusion devices. In the preferred embodiment, there is a proximal balloon and an optional distal balloon. FIG. 1E shows a sheath or steerable catheter 116 with a proximal balloon 120 (deflated) and distal balloon 121 (deflated). Proximal balloon 120 encircles the outer diameter of the catheter and is inflated from outside the body via lumen 122. Optionally, there may be a pilot balloon 123 located on the proximal end of lumen 122 to provide a visual indication of the pressure in balloon 120. Distal balloon 121 may be inflated from outside the body via lumen 124. The distal balloon may have its own dedicated deployment lumen 125 which is attached to the OD of catheter 116 and is slidably connected with lumen 124; alternately, the system may eliminate the need for lumen 125 by instead having the distal balloon be inflated via lumen 124 which is deployed through the ID of catheter 116 instead of a dedicated lumen or by having it be inflated via lumen 124 which is fixedly connected to the OD of catheter 116. Alternately still, the system may eliminate the need for lumen 124 by having the distal balloon be attached to an extension of lumen 122 and inflating the distal balloon simultaneously with the proximal balloon.

FIG. 1F shows the catheter 116 inside the bronchial airways 126 with the proximal balloon 120 and distal balloon 121 both inflated within the airways, thereby limiting the flooded area 127. Instruments may be deployed through the catheter inner diameter 128 into the flooded airway. Additionally or alternately, imaging instruments may be deployed into the flooded airway prior to flooding to aid in balloon placement or the flooding process.

In alternate embodiments, one or both occlusion devices may be a non-balloon component, such as a flap, expanding device, or one-way valve. FIG. 1G shows an example embodiment with a proximal expanding device and a distal expanding device. A proximal expandable cone 129 is incorporated into the outer diameter of catheter 116. The distal occlusion device 130 is also an expandable cone and is delivered into the appropriate position in the airway 126 via a delivery mechanism 131. The delivery mechanism may be released from the deployed distal cone and removed from the catheter; alternately, the delivery mechanism may stay connected to the distal cone and it would also be used as the recovery mechanism for the distal cone at the end of the procedure. Both the proximal and distal expandable cones may be expanded on command by any of the following methods: soft polymer or shape memory strands or sheets are compressed axially by a sleeve or locking mechanism which is then released to allow expansion into a preset cone shape, shape memory or piezoelectric strands or sheets are preset to exhibit an axially compressed shape at deployment conditions (e.g., body temperature or zero current) and then are activated by changing the temperature or current past a defined threshold, or soft material allows for low-friction passage along the airways until fluid is introduced creating pressure pinning the edges of the soft material against the airway walls and thereby creating an obstruction to further fluid filling. FIG. 1H-1I illustrate the last-mentioned embodiment. FIG. 1H shows that prior to flooding, the proximal cone 132 and distal cone 133 are soft enough to easily deflect anytime they contact the airway wall. The distal cone may be connected to the catheter via a guidewire 133. FIG. 1I shows that when the fluid 127 is introduced into the lung through the catheter, it pressurizes the interior of the proximal cone 132 and distal cone 133 causing firm contact with the vessel wall and preventing fluid egression into connecting lung areas. The pressurization caused by the fluid is represented by pressure lines 135.

In any of these embodiments, the distal occlusion device may be an optional feature, only used if the distal extent of airway flooding needs to be controlled, such as in the instance where the lesion is more centrally located and to fill the lung to the periphery would be too time-consuming or expensive.

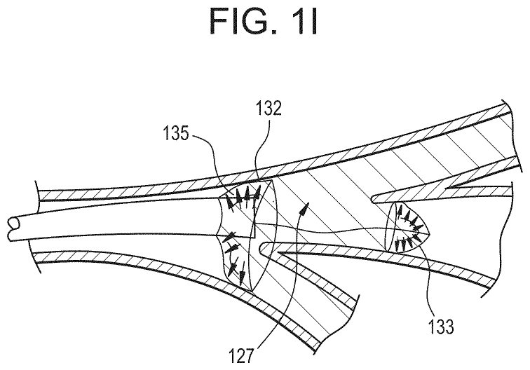

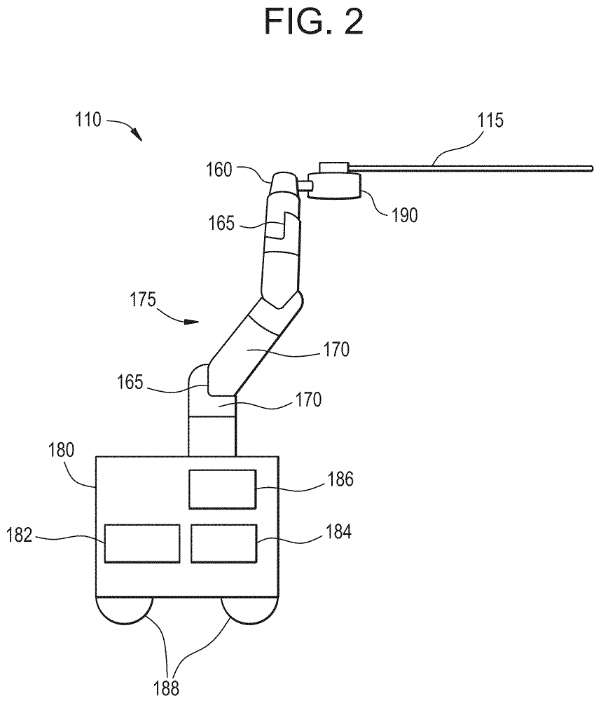

The above embodiments show the occlusion devices integrated into a catheter, but the occlusion devices may alternately be integrated into the bronchoscope, into the biopsy sheath or tool, or into an intermediate device that is deployed through the bronchoscope or catheter prior to the deployment of the biopsy tool. Deployment of the occluding devices may be manually or robotically controlled. FIG. 2 illustrates an example robotic arm 175 of a surgical robotic system 110 for guiding instrument movement in through the luminal network 140 of FIG. 1B. The surgical robotic system 110 includes a base 180 coupled to one or more robotic arms, e.g., robotic arm 175. The robotic am1 175 includes multiple arm segments 170 coupled at joints 165, which provide the robotic arm 175 multiple degrees of freedom. As an example, one implementation of the robotic arm 175 may have seven degrees of freedom corresponding to seven arm segments. In some embodiments, the robotic arm 175 includes set up joints that use a combination of brakes and counterbalances to maintain a position of the robotic arm 175. The counterbalances may include gas springs and/or coil springs. The brakes, e.g., fail safe brakes, may include mechanical 311d/or electrical com-ponents. Further, the robotic arm 175 may be a gravity-assisted passive support type robotic arm.

The robotic arm 175 may be coupled to an instrument device manipulator (IDM) 190 using a mechanism changer interface (MCI) 160. The IDM 190 may be removed and replaced with a different type of IDM, for example, a first type of IDM configured to manipulate an endoscope, or a second type of IDM configured to manipulate a laparoscope. The MCI 160 includes connectors to transfer pneumatic pressure, electrical power, electrical signals, and optical signals from the robotic arm 175 to the IDM 190. The MCI 160 may be a set screw or base plate connector. The IDM 190 manipulates surgical instruments, for example the endoscope 115 using techniques including direct drive, harmonic drive, geared drives, belts and pulleys, magnetic drives, and the like. The MCI 160 is interchangeable based on the type of IDM 190 and may be customized for a certain type of surgical procedure. The robotic arm 175 may include joint level torque sensing capabilities (e.g., using one or more torque sensors positioned at or near the joints 165) and a wrist at a distal end.

Robotic arm 175 of the surgical robotic system 110 may manipulate the endoscope 115 using elongate movement members. The elongate movement members may include pull wires, also referred to as pull or push wires, cables, fibers, or flexible shafts. For example, the robotic arm 175 may actuate multiple pull wires coupled to the endoscope 115 to deflect the tip of the endoscope 115. The pull wires may include both metallic and non-metallic materials, for example stainless steel, Kevlar, tungsten, carbon fiber, and the like. The endoscope 115 may exhibit nonlinear behavior in response to forces applied by the elongate movement members. The nonlinear behavior may be based on stiffness and compressibility of the endoscope 115, as well as variability in slack or stiffness between different elongate movement members.

The base 180 may be positioned such that the robotic arm 175 has access to perform or assist with a surgical procedure on a patient, while a user such as a physician may control the surgical robotic system 110 from the comfort of the command console. In some embodiments, the base 180 may be coupled to a surgical operating table or bed (e.g., a platform 102) for supporting the patient. The base 180 may be communicatively coupled to the command console 105 shown in FIG. 1A.

The base 180 may include a source of power 182, pneumatic pressure 186, and control and sensor electronics 184-including components such as a central processing unit, data bus, control circuitry, and memory-and related actuators such as motors to move the robotic arm 175. As used herein, the term “actuator” may refer to a mechanism for physically adjusting the position and/or orientation of the robotic arm 175. The electronics 184 may implement the biopsy guidance techniques described herein. The electronics 184 in the base 180 may also process and transmit control signals communicated from the command console. In some embodiments, the base 180 includes wheels 188 to transport the surgical robotic system 110 and wheel locks/brakes (not shown) for the wheels 188. Mobility of the surgical robotic system 110 helps accommodate space constraints in a surgical operating room as well as facilitate appropriate positioning and movement of surgical equipment. Further, the mobility allows the robotic arm 175 to be configured such that the robotic arm 175 does not interfere with the patient, physician, anesthesiologist, or any other equipment. During procedures, a user may control the robotic arm 175 using control devices, for example the command console.

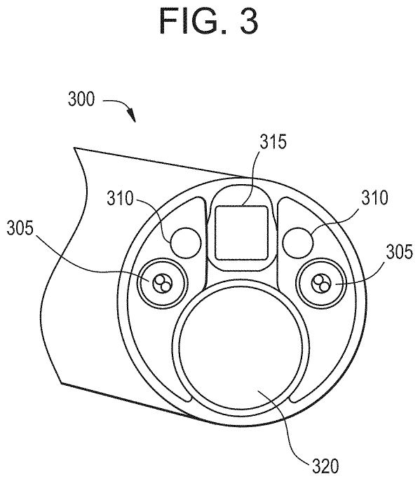

FIG. 3 illustrates the distal end 300 of an example endoscope having imaging and EM sensing capabilities as described herein, for example the endoscope 115. As shown in FIG. 3, the distal end 300 of the endoscope includes an imaging device 315, illumination sources 310, and may include ends of EM sensor coils 305. The distal end 300 further includes an opening to a working channel 320 of the endoscope through which surgical instruments, such as biopsy needles, cytology brushes, and forceps, may be inserted along the endoscope shaft, allowing access to the area near the endoscope tip.

The illumination sources 310 provide light to illuminate a portion of an anatomical space. The illumination sources may each be one or more light-emitting devices configured to emit light at a selected wavelength or range of wavelengths. The wavelengths may be any suitable wave length, for example visible spectrum light, infrared light, x-ray (e.g., for fluoroscopy), to name a few examples. In some embodiments, illumination sources 310 may include light-emitting diodes (LEDs) located at the distal end 300. In some embodiments, illumination sources 310 may include one or more fiber optic fibers extending through a length of the endoscope to transmit light through the distal end 300 from a remote light source, for example an X-ray generator. Where the distal end 300 includes multiple illumination sources 310 these may each be configured to emit the same or different wavelengths of light as one another.

The imaging device 315 may include any photo sensitive substrate or structure configured to convert energy representing received light into electric signals, for example a charge-coupled device (CCD) or complementary metal oxide semiconductor (CMOS) image sensor. Some examples of imaging device 315 may include one or more optical fibers, for example a fiber optic bundle, configured to transmit an image from the distal end 300 of the endoscope to an eyepiece and/or image sensor at the proximal end of the endoscope. Imaging device 315 may additionally include one or more lenses and/or wavelength pass or cutoff filters as required for various optical designs. The light emitted from the illumination sources 310 allows the imaging device 315 to capture images of the interior of a patient's luminal network. These images may then be transmitted as individual frames or series of successive frames (e.g., a video) to a computer system such as command console 200 for processing as described herein.

Electromagnetic coils 305 located on the distal end 300 may be used with an electromagnetic tracking system to detect the position and/or orientation of the distal end 300 of the endoscope while it is disposed within an anatomical system. In some embodiments, the coils 305 may be angled to provide sensitivity to electromagnetic fields along different axes, giving the disclosed navigational systems the ability to measure a full 6 degrees of freedom: three positional and three angular. In other embodiments, only a single coil may be disposed on or within the distal end 300 with its axis oriented along the endoscope shaft of the endoscope. Due to the rotational symmetry of such a system, it is insensitive to roll about its axis, so only 5 degrees of freedom may be detected in such an implementation.

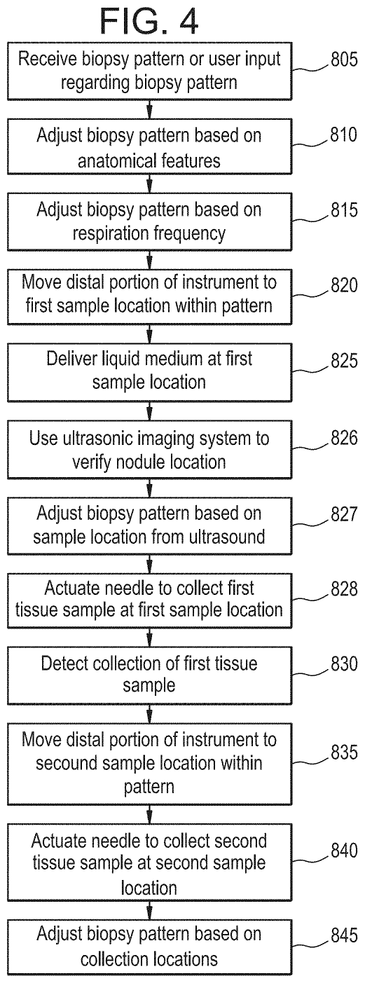

In accordance with one or more aspects of the present disclosure, FIG. 4 depicts a flow chart of an exemplary process in accordance with the present disclosure.

In accordance with one or more alternative exemplary embodiments, the present disclosure may be utilized for modalities. At block 805, a biopsy pattern or a user input regarding a biopsy pattern is received. At block 810, the biopsy pattern is adjusted based on anatomical features. At block 815, the biopsy pattern is adjusted based on respiration frequency. At block 820, the distal portion of the instrument is moved to the first sample location within the biopsy pattern. At block 825, the liquid medium is delivered to the first sample location. At block 826, the ultrasonic imaging system is utilized to verify nodule location. At block 827, the biopsy pattern is adjusted based upon the sample location from the ultrasound image. At block 828, a biopsy needle is actuated to collect a first tissue sample at the first target location. At block 830, collection of the first tissue sample is detected. At block 835, the distal portion of the instrument is moved to a second sample location within the biopsy pattern. At block 840, the biopsy needle is actuated to collect a second tissue sample at the second sample location. At block 845, the biopsy pattern is adjusted based on collection locations. Throughout the steps in blocks 828 to 845, the ultrasound imaging system may be used to verify the trajectory and position of the biopsy needle.

Although the embodiments of the process of FIG. 4 are described with respect to the collection of samples at two biopsy locations, similar steps may be conducted for any number of sample locations.

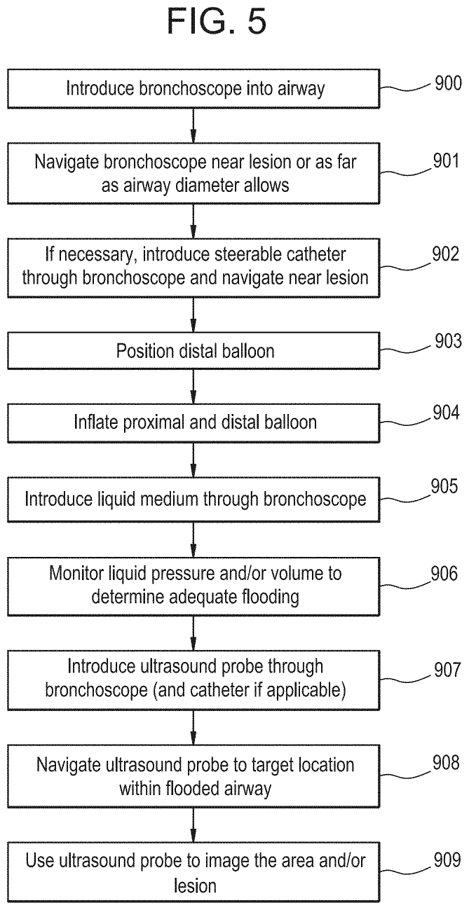

In accordance with one or more aspects of the present disclosure, FIG. 5 depicts a flow chart of an exemplary subprocess, involving occluding portions of the airway, introducing liquid, and introducing instruments, in accordance with the present disclosure. The blocks within FIG. 5 are subprocesses of the process blocks 820, 825, and 826 in FIG. 4. The introduction and navigation of instruments and liquid into the airways may be done manually or robotically.

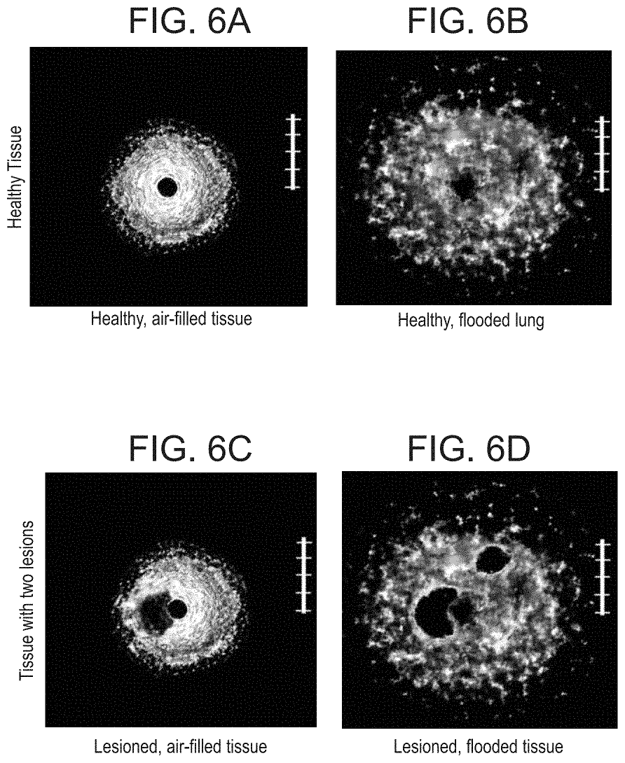

FIG. 6A-6D are representative figures illustrating the difference between ultrasound images of air-filled lung tissue (FIG. 6A & FIG. 6C) and flooded lung tissue (FIG. 6B & FIG. 6D).

FIGS. 6A and 6B show representative radial ultrasound images of the same location in the same healthy lung tissue, with FIG. 6A representing an air-filled lung and FIG. 6B representing a flooded lung. The air-filled lung in FIG. 6A creates a ‘snowstorm’ effect because of the multiple air-tissue boundaries. These boundaries cause ultrasound reflections which then reverberate back and forth between the boundary and the transducer and are visualized on the ultrasonic image as hyperechoic repetition artifacts. Boundaries between different types of healthy lung tissue, such as airway cartilage next to soft parenchymal tissue, are lost due to the reverberations and significant signal attenuation caused at air boundaries. The ultrasound of healthy, flooded lung tissue (FIG. 6B) has a deeper penetration of signal for the same frequency of ultrasound because the air-tissue interfaces are replaced with less-attenuating fluid-tissue interfaces, which also allows for structures of different tissue composition (such as airways and vessels) to be better distinguished.

The above-mentioned considerations affect ultrasound images when lesions are in the vicinity of the ultrasound transducer, and this is shown in FIGS. 6C and 6D which are representative of images that may be obtained of the same location of the same lesion-filled lung tissue, with FIG. 6C representing an air-filled lung and FIG. 6D representing a flooded lung. In air-filled lung tissue, only solid lesions that are concentric, eccentric, or adjacent to the radial probe are detectable; lesions separated from the probe by a layer of air will not be imaged due to the reverberation and attenuation generated at the tissue-air interface. Liquid-filled lung tissue removes the air-tissue boundaries and allows for non-adjacent lesions to be imaged, as shown in the upper right of the ultrasound image in FIG. 6D; this same lesion would not be detectable in the air-filled lung of FIG. 6C.

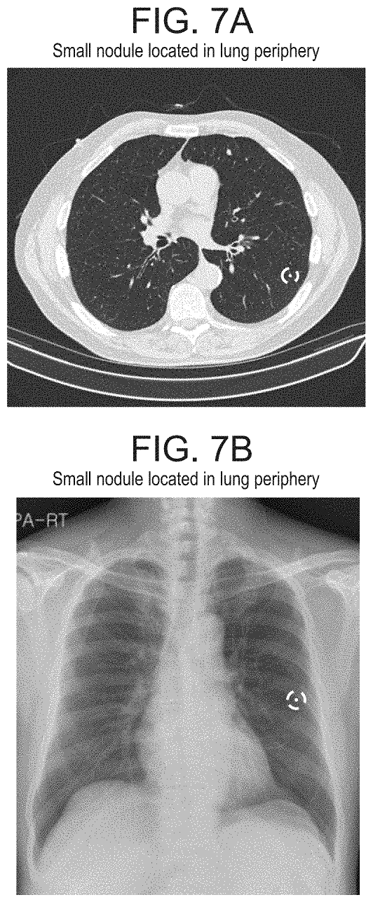

FIGS. 7A-7B are representations of x-ray and CT images of a small nodule located in the periphery of the lung. In conventional x-ray systems, a beam of x-rays is directed through an object such as the human body onto a flat x-ray photographic film. The beam of x-rays is selectively absorbed by structures within the object, such as bones within the human body. Since the exposure of the x-ray film varies directly with the transmission of x-rays through the body (and varies inversely with the absorption of x-rays), the image that is produced provides an accurate indication of any structures within the object that absorbed the x-rays. As a result, x-rays have been widely used for non-invasive examination of the interior of objects and have been especially useful in the practice of medicine. FIG. 7B is a representation of an x-ray image of an identified tissue abnormality in the periphery of the left lung.

Many of the limitations of conventional x-ray systems may be avoided by x-ray computer tomography, which is often referred to as CT. In particular, CT provides three-dimensional views and the imaging of structures and features that are unlikely to be seen very well in a conventional x-ray. A CT scanning equipment typically includes a computer, a large toroidal structure and a platform that is movable along a longitudinal axis through the center of the toroidal structure. Mounted within the toroidal structure are an x-ray source (not shown) and an array of x-ray detectors (not shown). The x-ray source is aimed substantially at the longitudinal axis and is movable around the interior of the toroidal structure in a plane that is substantially perpendicular to the longitudinal axis. The x-ray detectors are mounted all around the toroidal structure in substantially the same plane as the x-ray source and are aimed at the longitudinal axis. To obtain a CT x-ray image, a patient is placed on the platform and the platform is inserted into the center of the toroidal structure. The x-ray source then rotates around the patient continuously emitting x-rays and the detectors sense the x-ray radiation that passes through the patient. Since the detectors are in the same plane as the x-ray source, the signals they receive relate essentially to a slice through the patient's body where the plane of the x-ray source and detectors intersect the body. The signals from the x-ray detectors are then processed by the computer to generate an image of this slice known in the art as an axial section.

As an example, x-rays may be emitted continuously for the full 360° around the patient and numerous features are observed but the overall approach is generally the same. While the patient remains motionless, the platform is moved along the longitudinal axis through the toroidal structure. In the course of this movement, x-ray exposures are continuously made of the portion of the patient on which CT is to be performed. Since the table is moving during this process, the different x-ray exposures are exposures of different slices of the portion of the patient being examined and the images generated by the computer are a series of axial sections depicting in three dimensions the portion of the patient's body that is being examined. The spacing between adjacent CT sections depends on the minimum size of the features to be detected. For detection at the highest resolution, center-to-center spacing between adjacent sections should be on the order of less than 2 mm. Using currently available imaging systems, the minimum detectable size of a potentially cancerous nodule in an axial section of the lung is about 2 mm ( 1/10 of inch), a size that is potentially treatable and curable if detected. FIG. 7A is a representation of a CT image of the same identified tissue abnormality in the periphery of the left lung as shown in FIG. 7B.

As previously stated, a preoperative procedure may be performed to take CT scans of a patient's lungs, and a computing system may use data from these scans to build a 3D model of the lungs of the patient. Such a model may provide 3D information about the structure and connectivity of the lung luminal network, including the topography and/or diameters of patient airways in some examples, as well as the estimated location relative to the lung luminal network of one or more lung nodules targeted for biopsy. Then, this model of the luminal network and nodule location(s) may be used by an endoscopic biopsy system to navigate to a target tissue site wherein one or more nodules are located.

Due to multiple potential factors, the actual real-time location of one or more nodules may differ from their estimated locations as described in a 3D model of the lungs. The lungs comprise highly dynamic, elastic tissue structures that vary considerably in size when fully inflated compared to when fully deflated. Therefore, it is important to understand that the location of one or more nodules targeted for biopsy as described in a 3D model of the lungs as obtained from a preoperative CT scan is merely an estimation of the real-time location of said nodule(s) and, as a consequence, the actual location(s) of one or more nodules targeted for biopsy may slightly change due to the compliant nature of the lung. The potential deviation in real-time nodule positioning from estimated location increases from centrally-located nodules to peripherally-located nodules. For larger lung nodules (for example, nodules greater than 12 mm in diameter) and/or lung nodules located more centrally (i.e. not located near the periphery of the lungs), the potential deviation in nodule positioning from its estimated location as described in the 3D model of the lungs tends to be relatively small and thus has little effect on the rate of successful biopsy.

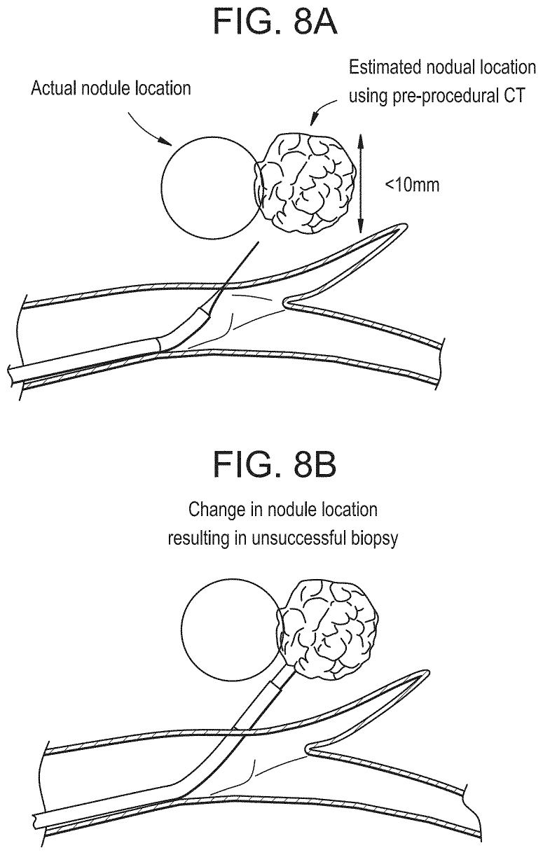

However, for small- to intermediate-sized nodules (<12 mm in diameter) located in the periphery of the lung, the potential deviation in nodule positioning from its estimated location as described in the 3D model of the lungs may be greater than the diameter of the nodule itself. Current endobronchial biopsy systems have no means of determining the real-time location of lung nodules and, as a result, an endoscopic biopsy system may fail to retrieve a tissue sample for the purposes of a cancer diagnosis from a nodule located in the periphery of the lung despite successfully deploying a biopsy needle into the estimated location of one or more nodules targeted for biopsy. This can have disastrous consequences for patients; biopsy errors such as these would lead to false negative cancer diagnoses and could potentially delay the initiation of cancer treatment. Additionally, the 5-year survival rate for patients diagnosed with lung cancer is significantly better for patients with Stage 1 lung cancer when compared to patients who receive cancer diagnoses at later stages. The earlier lung cancer is detected, diagnosed, and treated, the better the patient outcomes. However, as described previously, diagnosis of early-stage lung cancer is difficult to achieve due to the small size of lung nodules at earlier stages. FIGS. 8A and 8B are representative depictions of a small- to intermediate-sized nodule located in the periphery of the lung that has shifted slightly in position from its location as initially imaged in a preprocedural CT scan. Since the endobronchial biopsy system cannot determine the real-time position of the nodule and correct for any deviations, the biopsy needle is advanced into the estimated location for the targeted nodule, missing the nodule entirely. Currently, there are no means to detect this error.

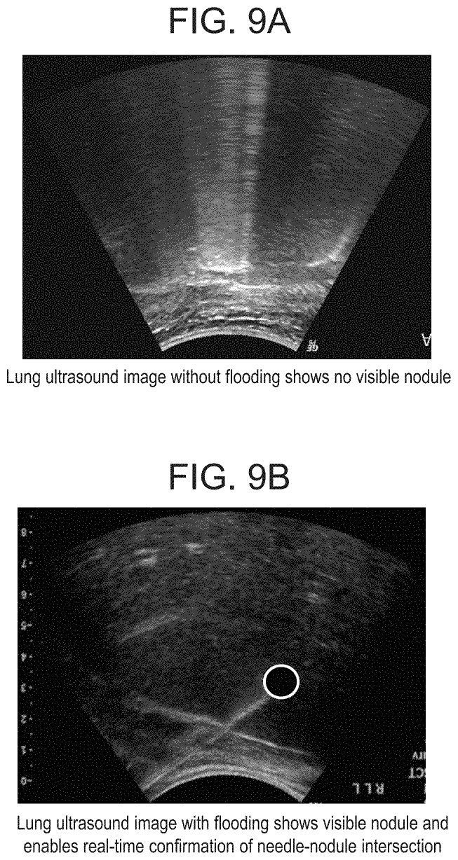

FIGS. 9A-9B are representations of ultrasound images of a small nodule targeted for endobronchial biopsy before and after localized filling of the lung with liquid medium. Note that both the biopsy needle and the lung nodule targeted for biopsy are not visible on the ultrasound image prior to the introduction of liquid media. After the introduction of liquid media, structures of different tissue composition (such as airways and vessels) are now better distinguished on the ultrasound image. Both the biopsy needle and nodule targeted for biopsy are now visualized, and real-time confirmation of successful introduction of the biopsy needle into the targeted nodule can now be achieved.

Additional imaging modalities exist or are emerging for imaging of lung tissue from the airway or other location outside of the nodule, as alternates to ultrasound. The presence of gas in the airways and parenchyma affects the resolution and depth of measurements in these cases.

Electrical impedance of tissue may be measured locally by injecting current through two electrodes and measuring the resultant voltage across two electrodes. Conversely, measurements may be taken by inducing a voltage and measuring the resulting current. Electrical bioimpedance arrays may be used to generate a 3D map of impedance values, mapping beyond the tissue that is contacting the electrodes; therefore, the impedance may be mapped using electrodes located inside the airways to characterize the parenchymal tissue beyond the airway. A limitation, however, is that the air inside the airways and the parenchyma acts as an electrical insulator and will not allow characterization of the tissue bioimpedance beyond the region of air.

A variety of light-based imaging techniques are used on the human body and have applications within the lungs. These technologies use the principles of reflection, refraction, absorption, scattering, and interference to image tissue. Spectroscopy may be used to analyze the tissue response to different light frequencies. Many optical sensing technologies in the lung are detrimentally affected by the presence of air because the readings are affected by strong backscattering due to large refractive-index mismatch between alveoli walls and enclosed air-filled regions.

Tissues of different compositions have different thermal properties, and these may be measured to create a map of the region of interest. Thermal-based measurements are affected by the thermal insulation properties of air.

Analysis of tissue interaction with sound waves can characterize many properties of the tissue, such as acoustical impedance mismatches at boundaries, speed of sound in the tissue, acoustic resonance, and elastic properties. The most common acoustic interrogation of tissue is ultrasound, however sound waves outside of the ultrasound range are also able to be employed. Air in the lungs affects both the speed of sound in the air-filled region (speed will be fastest in this region) and the impedance mismatch (which will be large between air and tissue).

Combinations of different imaging modalities are often used for imaging in the human body. An example of a dual-modality imaging technique is photoacoustics. Because one or more of the combination technologies are often based on bioimpedance, optics, thermal, or acoustic measurement systems, the air in the lung will affect the ability of the system to accurately and effectively characterize the tissue in the region of interest.

In summary, essentially, any energy-based imaging technology will be affected (often negatively) by the presence of air.

All of the listed imaging techniques may be used, in addition to providing a map to allow correct placement of biopsy tools, to characterize potentially cancerous tissue, which may have a different composition than that of the surrounding lung tissue. Characterizing potentially cancerous tissue is useful because it may allow for the correct section of the nodule to be targeted for biopsy and can provide in situ diagnostic information about the nature of the nodule.

Similar to the description regarding ultrasound, with other imaging techniques the imaging system source and detection components may be located on the distal end of a bronchoscope, on a tool that is inserted through the bronchoscope, or on a standalone tool. The preselected region of the bronchial tree is flooded, as described in the ultrasound embodiment, and then the imaging tool is used to create a map of the tissue of interest. The fluid eliminates, or very nearly eliminates, all gas inside the lung region of interest, thereby removing a source of limitation for the imaging system and providing more complete and accurate tissue information for biopsy. Once the biopsy plan is updated based on the imaging results, a biopsy tool is used to collect a tissue sample. The imaging system may also be used to visualize the biopsy tool as it advances to the biopsy site and to confirm that the biopsy is taken from the correct place.

The goal of a biopsy is to identify the composition of the nodule. This goal may be accomplished without needing to remove tissue from the lung, thereby inflicting less trauma on the patient, if there is sufficient evidence gathered in situ to accurately identify the tissue composition. The above imaging technologies may be used to provide this information about the region of issue, quantifying the differences between healthy and unhealthy tissues.

In this embodiment, the lung flooding will take place in the same manner as above; however, a biopsy tool will not be needed. The imaging will identify the composition of the tissue of interest; thus, informing the clinical plan of action for that patient.

In an alternate or complementary embodiment, when the fluid is drained after the imaging, biopsy, and/or treatment portion of the procedure, it is collected and analyzed for the presence of tumor biomarkers. Alternately, the lung flooding, draining and collection, and fluid analysis may comprise the entire procedure with no additional imaging or biopsy steps. An advantage of this technique is that the fluid provides a non-traumatic method of collecting biopsy information on all areas of the lung tissue that the fluid was in contact with.

In-situ treatments for lung cancer may be performed when surgical resection is contraindicated and in particular, treatments via an endobronchial approach are tissue sparing and less invasive.

Some of the common treatments employ energy delivery to the tumor site in order to destroy tumor cells. These include high intensity focused ultrasound, electroporation, cryoablation, radiofrequency ablation, microwave ablation, laser thermal therapy, reversible electroporation with chemotherapeutic delivery, irreversible electroporation, and others. The properties of air limit the effectiveness of any of these techniques and can result in damage to healthy tissue when the energy is effectively localized to the tumor site.

Tumors may be treated by directing treatment drugs to the affected area. Targeted delivery can increase the efficacy of the drug treatment and decrease negative side effects.

In the case of enacting ablation of tumor cells via energy delivery, lung flooding takes place in the same manner as the above to remove regions of air, and then the energy delivery is performed.

In cases of ablation where an invasive probe is utilized, the fluid is used to more uniformly spread the energy delivery within the localized fluid region, for example, acting as an electrical or thermal coupling mechanism. The energy would more comprehensively target the tumor site compared to a lung with residual gas and would be confined to that are, causing less damage to the surrounding healthy tissue.

In cases of non-invasive ablation, such as HIFU, the de-gassed lung creates a low attenuation beam path allowing the energy to be delivered to the tumor and converted into thermal energy. The fluid provides accessibility into the lung previously unavailable due to high attenuation of energy due to the presence of gas-filled alveoli and airways. The tumor, and not the flooded lung tissue, converts the energy into a thermal dose and therefore heats only the selected tumor mass; this spares healthy tissue, retains lung function, and puts less trauma on the patient.

In cases of electroporation, the fluid removes the pockets of electrically-insulative air from the lung, thereby enabling a more uniform region of electric field and more uniform formation of nanopores in the targeted tissue cells. Irreversible electroporation (IRE) has been shown to be less effective in the lung than other organs and it is hypothesized to be because of the heterogeneity of lung as well as the presence of insulating air pockets; lung flooding will help to alleviate these obstacles.

The present disclosure comprises at least the following aspects:

Aspect 1. A method for endobronchial ultrasonic imaging of nodules within the lung, the method comprising the steps of: filling preselected pulmonary air passages proximate pulmonary tissues containing one or more nodules targeted for biopsy with a liquid medium; introducing an ultrasonic imaging device into the lungs; and transmitting ultrasonic energy through the liquid medium for visualizing one or more nodules.

Aspect 2. The method of Aspect 1, further comprising performing a biopsy of the one or more nodules based upon, at least in part, the visualizing of one of more nodules using ultrasonic energy.

Aspect 3. The method any one of Aspects 1-2, further comprising removing the liquid medium from the lungs.

Aspect 4. The method any one of Aspects 1-3, wherein the liquid medium comprises a perfluorochemical.

Aspect 5. A method for collecting one or more samples from a target tissue site of a patient, the method comprising: through a user interface of a robotic medical system, receiving a user input that selects one or more samples within a target tissue site for which collection is desired; moving a distal portion of an instrument of the robotic medical system to a sample location adjacent the target tissue site; introducing a liquid medium at the sample location; transmitting ultrasonic energy through the liquid medium for visualizing one or more samples within the target tissue site; and guiding the instrument to obtain at least one tissue sample from the target tissue site.

Aspect 6. The method any one of Aspect 5, further comprising adjusting a position of a distal portion of the instrument of the robotic medical system after visualizing one or more samples within the target tissue site based on, at least in part, a user input.

Aspect 7. The method any one of Aspects 5-6, wherein adjusting the position of the distal portion of the instrument of the robotic medical system is based on, at least in part, a determined displacement of a location of one more samples within the target tissue site from a preprocedural model representative of the location of one or more samples within the target tissue site.

Aspect 8. The method any one of Aspect 5-7, further comprising adjusting one or more penetration depths, one or more sampling velocities, one or more sampling intervals, or one or more sampling forces of the instrument when guiding the instrument to obtain at least one sample from the target tissue site.

Aspect 9. The method any one of Aspects 5-8, wherein adjusting one or more penetration depths, one or more sampling velocities, one or more sampling intervals, or one or more sampling forces of the instrument when guiding the instrument to obtain at least one sample from the target tissue site is based on, at least in part, a determined displacement of a location of one more samples within the target tissue site from a preprocedural model representative of the location of one or more samples within the target tissue site.

Aspect 10. The method any one of Aspects 5-9, further comprising removing the liquid medium from the lungs.

Aspect 11. The method any one of Aspects 5-10 wherein the liquid medium comprises a perfluorochemical.

Aspect 12. A system configured to aid in obtaining a set of one or more biopsy samples from a tissue site, the system comprising: an instrument through which the set of one or more biopsy samples can be collected, the instrument including a fluid channel and an ultrasound transducer element; an actuator configured to control movements of the instrument; at least one computer-readable memory having stored thereon executable instructions; and one or more processors in communication with the at least one computer-readable memory and configured to execute the instructions to cause the system to at least: access a biopsy pattern comprising one or more sample locations within the tissue site; calculate movement of the instrument according to the biopsy pattern; move the instrument to one or more positions corresponding to the one or more sample locations. introduce, via the fluid channel, a liquid medium at the one or more sample locations; and transmit ultrasonic energy through the liquid medium for visualizing a target tissue site.

Aspect 13. The system of Aspect 12, further comprising a user input device configured to receive the biopsy pattern, a command to access the biopsy pattern, or a command to calculate movement of the instrument according to the biopsy pattern.

Aspect 14. The system any one of Aspects 12-13, further comprising at least one localization sensor in the instrument.

Aspect 15. The system any one of Aspects 12-14, wherein the localization sensor and the ultrasound transducer element enable capture of ultrasound slice data and a position and orientation of the ultrasound transducer at each acquired slice.

Aspect 16. The system any one of Aspects 12-15, wherein a three dimensional tissue structure model is producible from the data acquired from the ultrasound transducer element and the localization sensor.

Aspect 17. The system any one of Aspects 12-16, further comprising a user interface screen configured to show the biopsy pattern.

Aspect 18. The system any one of Aspects 12-17, wherein the one or more processors are configured to execute the instructions to cause the system to at least: adjust the biopsy pattern or a route representing the movement of the instrument to the one or more positions based on information received from a user.

Aspect 19. The system any one of Aspects 12-18, further comprising a set of one or more location sensors; and wherein the one or more processors are configured to execute the instructions to cause the system to at least: calculate (1) at least one position of the set of location sensors or (2) a position of a distal end of the instrument based on a data signal from the set of one or more location sensors; and control movement to the one or more positions based on the calculated position.

Aspect 20. The system any one of Aspects 12-19, wherein the instrument comprises: a scope configured to reach the tissue site; and a collection device configured to (1) be removably placed within the scope or (2) pass through the scope and collect the set of one or more biopsy samples.

Aspect 21. The system any one of Aspects 12-20, wherein the one or more processors are further configured to execute the instructions to cause the system to at least: position the instrument to a first position, confirm receiving a first sample, and position the instrument to a second position in response to a confirmation of receiving the first sample.

Aspect 22. The system any one of Aspects 12-21, wherein the instrument comprises a collection device configured to obtain the set of one or more biopsy samples; wherein the actuator is configured to control movements of the collection device; wherein the collection device further comprises a marker at a distal end of the collection device; and wherein the one or more processors are further configured to execute the instructions to cause the system to at least: determine movement of the collection device according to a movement of the marker; and adjust the one or more sample locations according to the movement of the collection device.

Aspect 23. The system any one of Aspects 12-22, wherein the biopsy pattern comprises one or more sample positions arranged in at least two dimensions.

Aspect 24. The system any one of Aspects 12-23, wherein the biopsy pattern comprises one or more sample positions arranged in a shape fitted to a shape of the tissue site.

Aspect 25. The system any one of Aspects 12-24, wherein the biopsy pattern further comprises one or more penetration depths, one or more sampling velocities, one or more sampling intervals, or one or more sampling forces corresponding to the one or more sample positions.

Aspect 26. The system any one of Aspects 12-25, wherein the one or more processors are further configured to execute the instructions to cause the system to adjust a position of a distal portion of the instrument based on, at least in part, a user input.

Aspect 27. The system any one of Aspects 12-26, wherein the one or more processors are further configured to calculate a displacement of a location of one more samples within the target tissue site from a preprocedural model representative of the location of one or more samples within the target tissue site.

Aspect 28. The system any one of Aspects 12-27, wherein the one or more processors are configured to execute the instructions to cause the system to at least: adjust the biopsy pattern or a route representing the movement of the instrument to the one or more positions based on, at least in part, a calculated displacement of the location of one more samples within the target tissue site from a preprocedural model representative of the location of one or more samples within the target tissue site.

Aspect 29. The system any one of Aspects 12-28, wherein the one or more processors are further configured to adjust one or more penetration depths, one or more sampling velocities, one or more sampling intervals, or one or more sampling forces corresponding to one or more sample positions based on, at least in part, a determined displacement of the location of one more samples within the target tissue site from a preoperative model representative of the location of one or more samples within the tissue site.

Aspect 30. The system any one of Aspects 12-29 wherein the instrument is further configured for removing, via the fluid channel, the liquid medium from one or more sample locations.

Aspect 31. The system any one of Aspects 12-30, wherein the liquid medium comprises a perfluorochemical.

Aspect 32. A robotic medical system, comprising: an instrument driver comprising a motor and an instrument interface, the instrument interface comprising a drive element operatively coupled to the motor; a steerable catheter comprising: an elongate body defining a lumen, a fluid channel extending through the elongate body to a distal end portion of the steerable catheter, the fluid channel configured for introducing a liquid medium a control element extending through the elongate body to a distal end portion of the steerable catheter, and an instrument base operatively coupled to the instrument interface, the instrument base comprising a pulley operatively coupled to the control element and the drive element such that actuation of the drive element by the motor actuates the pulley causing actuation of the control element and the distal end portion; a controller having control logic configured to operate the motor of the instrument driver; and an ultrasound transducer integrated in the steerable catheter and configured to acquire data, wherein the data acquired from the ultrasound transducer during movement through a patient is used by the controller, based on the control logic, to control the motor of the instrument driver to actuate the distal end portion of the steerable catheter to navigate the steerable catheter through the patient.

Aspect 33. The robotic medical system of Aspect 32, further comprising at least one localization sensor in the steerable catheter.

Aspect 34. The robotic medical system any one of Aspects 32-33, wherein the localization sensor and the ultrasound transducer enable capture of ultrasound slice data and a position and orientation of the ultrasound transducer at each acquired slice.

Aspect 35. The robotic medical system any one of Aspects 32-34, wherein a three dimensional tissue structure model is producible from the data acquired from the ultrasound transducer and the localization sensor.

Aspect 36. The robotic medical system any one of Aspects 32-35, further comprising a user interface screen configured to show data acquired by the ultrasound transducer.

Aspect 37. The system any one of Aspects 32-36, wherein the controller is further configured to control the motor of the instrument driver to actuate the distal end portion of the steerable catheter based on, at least in part, a user input.

Aspect 38. The robotic medical system any one of Aspects 32-37, wherein the controller is further configured to calculate a displacement of the location of one more anatomical features from a preprocedural model representative of the location of one or more anatomical features based on, at least in part, data acquired from the ultrasound transducer.

Aspect 39. A system configured to navigate a luminal network of a patient, the system comprising: a field generator configured to generate an electromagnetic (EM) field; a steerable instrument comprising a set of one or more EM sensors at a distal end, a ultrasound transducer element, and a fluid channel; a set of one or more respiration sensors; at least one computer-readable memory having stored thereon executable instructions; and one or more processors in communication with the at least one computer-readable memory and configured to execute the instructions to cause the system to at least: access a preoperative model representative of the luminal network; access a mapping between a coordinate frame of the EM field and a coordinate frame of the preoperative model; calculate at least one position of the set of EM sensors within the EM field based on a data signal from the set of EM sensors; calculate at least one position of one or more anatomical features based on a data signal from the ultrasound transducer element; calculate a frequency of respiration of the patient based on a data signal from the set of one or more respiration sensors; and determine a position of the distal end of the steerable instrument relative to the preoperative model based on the mapping, the frequency of respiration, the at least one position of one or more anatomical features, and the at least one position of the set of EM sensors within the EM field.

Aspect 40. The system of Aspect 39, wherein the one or more processors are configured to execute the instructions to cause the system to at least: transform one or more data signals from the set of respiration sensors into a frequency domain representation of the one or more data signals; and identify the frequency of respiration from the frequency domain representation of the one or more data signals.

Aspect 41. The system any one of Aspects 39-40, wherein the one or more processors are configured to execute the instructions to cause the system to at least: apply a predictive filter to one or more data signals from the set of EM sensors, the predictive filter configured to predict respiration motion due to the respiration; and remove components of the one or more data signals attributable to the predicted respiration motion to determine the position of the distal end of the steerable instrument relative to the preoperative model.

Aspect 42. The system any one of Aspects 39-41, wherein the one or more processors are configured to execute the instructions to cause the system to at least calculate at least one magnitude of displacement of the set of one or more respiration sensors between an inspiration phase and an expiration phase of respiration of the patient.

Aspect 43. The system any one of Aspects 39-42, wherein the one or more processors are configured to execute the instructions to at least: determine at least one position of the set of EM sensors relative to the set of respiration sensors; calculate at least one positional displacement of the set of EM sensors between the inspiration and the expiration phases based on (i) the determined at least one position of the set of EM sensors relative to the set of respiration sensors and (ii) the at least one magnitude of displacement of the set of one or more respiration sensors between the inspiration phase and the expiration phase; and determine the position of the distal end of the steerable instrument relative to the preoperative model based on the calculated at least one positional displacement of the set of EM sensors between the inspiration phase and the expiration phase.

Aspect 44. The system any one of Aspects 39-43, wherein the set of one or more respiration sensors comprises a first additional EM sensor positioned, in use, at a first position on a body surface and a second additional EM sensor positioned, in use, at a second position of the body surface, wherein the second position is spaced apart from the first position such that a first magnitude of displacement of the first additional EM sensor is greater than a second magnitude of displacement of the second additional EM sensor between the inspiration phase and the expiration phase.

Aspect 45. The system any one of Aspects 39-44, wherein the one or more processors are configured to execute the instructions to cause the system to at least: estimate a movement vector for at least a portion of the preoperative model based on the calculated at least one magnitude of displacement; translate the preoperative model within the coordinate frame of the EM field based on the estimated movement vector; and determine a position of the distal end of the steerable instrument based on the translated preoperative model.

Aspect 46. The system any one of Aspects 39-45, further comprising a display, wherein the one or more processors are configured to execute the instructions to cause the system to at least: generate a graphical representation of the position of the distal end of the steerable instrument relative to the preoperative model; and render the generated graphical representation on the display.

Aspect 47. A system for ablating tissue, the system comprising: an elongate shaft having a movable distal portion and at least one fluid channel configured for introducing a liquid medium to a target tissue site; and an ablation element comprising an ultrasound transducer coupled to the movable distal portion of the elongate shaft, wherein the ultrasound transducer comprises a single ultrasound transducer element having an active portion and an inactive portion, wherein the ultrasound transducer is configured to sense a thickness of a target tissue, and wherein the ultrasound transducer is configured to deliver a collimated beam of ultrasound energy comprising ablation energy to ablate the target tissue without contacting the target tissue with the elongate shaft or any structure disposed thereon.

Aspect 48. The system of Aspect 47, further comprising a reflecting element operably coupled with the ultrasound transducer, the reflecting element redirecting the collimated beam of ultrasound energy emitted from the ultrasound transducer to change a direction or a pattern of the collimated beam of ultrasound energy.

Aspect 49. The system any one of Aspects 47-48, wherein the reflecting element is configured to move relative to the ultrasound transducer so that the collimated beam of ultrasound energy is emitted at varying angles or positions.

Aspect 50. The system any one of Aspects 47-49, further comprising a processor configured to adjust the collimated beam of ultrasound energy in response to the sensed thickness of the target tissue.

Aspect 51. The system any one of Aspects 47-50, wherein the processor is configured to adjust one or more of frequency, a voltage, a duty cycle, a pulse length, or a position of the collimated beam of ultrasound energy in response to the sensed thickness of the target tissue.

Aspect 52. The system any one of Aspects 47-51, further comprising a backing layer coupled to the ultrasound transducer, the backing layer configured to provide a heat sink for the ultrasound transducer.

Aspect 53. The system any one of Aspects 47-52, wherein the backing layer comprises a plurality of grooves extending longitudinally along an outside wall of the backing layer.

Aspect 54. The system any one of Aspects 47-53, wherein the inactive portion comprises a material configured to conduct heat away from the active portion.

Aspect 55. The system any one of Aspects 47-54, further comprising a flow of fluid configured to cool the ultrasound transducer.

Aspect 56. A method for ablating tissue, said method comprising: providing an ablation system comprising: an elongate shaft and an ablation element comprising at least one ultrasound transducer, wherein the ultrasound transducer comprises a single ultrasound transducer element having an active portion and an inactive portion; and at least one fluid channel; positioning the ablation element adjacent a target tissue site; delivering, via at least one fluid channel of the ablation system, a liquid medium to the target tissue site to energetically couple, at least in part, at least one ultrasound transducer to a target tissue; sensing a thickness of the target tissue with the ultrasound transducer; and ablating at least a portion of the target tissue with a beam of ultrasound energy, thereby forming a zone of ablation comprising a continuous lesion in the target tissue.

Aspect 57. The method of Aspect 56, wherein delivering the beam of ultrasound energy comprises reflecting the beam of ultrasound energy off of a reflecting element operably coupled with the ultrasound transducer thereby redirecting the beam of ultrasound energy and changing a direction or pattern of the beam of ultrasound energy.

Aspect 58. The method any one of Aspects 56-57, wherein reflecting the beam of ultrasound energy comprises moving the reflecting element relative to the ultrasound transducer so that the beam of ultrasound energy is emitted at varying angles or positions.

Aspect 59. The method any one of Aspects 56-58, further comprising directing the beam of ultrasound energy along a path such that the zone of ablation in the target tissue has a ring shape, elliptical shape, linear shape, curvilinear shape, or combinations thereof.

Aspect 60. The method any one of Aspects 56-59, further comprising adjusting the beam of ultrasound energy in response to the sensed thickness of the target tissue.

Aspect 61. The method any one of Aspects 56-60, wherein adjusting the beam of ultrasound energy comprises adjusting one or more of frequency, a voltage, a duty cycle, a pulse length, or a position of the beam of ultrasound energy in response to the sensed thickness of the target tissue.

Aspect 62. The method any one of Aspects 56-61, further comprising cooling the ultrasound transducer.

Aspect 63. The method any one of Aspects 56-62, wherein the inactive portion comprises a material configured to conduct heat away from the active portion.

Aspect 64. The method any one of Aspects 56-63, wherein the ablation system further comprises a backing layer coupled to the ultrasound transducer, the backing layer configured to provide a heat sink for the ultrasound transducer, wherein the backing layer comprises a plurality of grooves extending longitudinally along an outside wall of the backing layer.

Although shown and described in what is believed to be the most practical and preferred embodiments, it is apparent that departures from specific designs and methods described and shown will suggest themselves to those skilled in the art and may be used without departing from the spirit and scope of the disclosure. The present disclosure is not restricted to the particular constructions described and illustrated but should be constructed to cohere with all modifications that may fall within the scope of the appended claims.

What is claimed is:

1. A method for endobronchial ultrasonic imaging of nodules within the lungs, the method comprising the steps of:
filling preselected pulmonary air passages proximate pulmonary tissues containing one or more nodules targeted for biopsy with a liquid medium;
introducing an ultrasonic imaging device into the lungs; and
transmitting ultrasonic energy through the liquid medium for visualizing one or more nodules.

2. The method of claim 1, further comprising performing a biopsy of the one or more nodules based upon, at least in part, the visualizing of one of more nodules using ultrasonic energy.

3. The method of claim 1, further comprising removing the liquid medium from the lungs.

4. The method of claim 1, wherein the liquid medium comprises a perfluorochemical.

5. A method for collecting one or more samples from a target tissue site of a patient, the method comprising:
through a user interface of a robotic medical system, receiving a user input that selects one or more samples within a target tissue site for which collection is desired;
moving a distal portion of an instrument of the robotic medical system to a sample location adjacent the target tissue site;
introducing a liquid medium at the sample location;
transmitting ultrasonic energy through the liquid medium for visualizing one or more samples within the target tissue site; and
guiding the instrument to obtain at least one tissue sample from the target tissue site.

6. The method of claim 5, further comprising adjusting a position of a distal portion of the instrument of the robotic medical system after visualizing one or more samples within the target tissue site based on, at least in part, a user input.

7. The method of claim 6, wherein adjusting the position of the distal portion of the instrument of the robotic medical system is based on, at least in part, a determined displacement of a location of one more samples within the target tissue site from a preprocedural model representative of the location of one or more samples within the target tissue site.

8. The method of claim 5, further comprising adjusting one or more penetration depths, one or more sampling velocities, one or more sampling intervals, or one or more sampling forces of the instrument when guiding the instrument to obtain at least one sample from the target tissue site.

9. The method of claim 8, wherein adjusting one or more penetration depths, one or more sampling velocities, one or more sampling intervals, or one or more sampling forces of the instrument when guiding the instrument to obtain at least one sample from the target tissue site is based on, at least in part, a determined displacement of a location of one more samples within the target tissue site from a preprocedural model representative of the location of one or more samples within the target tissue site.

10. The method of claim 5, further comprising removing the liquid medium from the lungs.

11. The method of claim 5, wherein the liquid medium comprises a perfluorochemical.

12. A system configured to aid in obtaining a set of one or more biopsy samples from a tissue site, the system comprising:
an instrument through which the set of one or more biopsy samples can be collected, the instrument including a fluid channel and an ultrasound transducer element;
an actuator configured to control movements of the instrument;
at least one computer-readable memory having stored thereon executable instructions; and
one or more processors in communication with the at least one computer-readable memory and configured to execute the instructions to cause the system to at least:
access a biopsy pattern comprising one or more sample locations within the tissue site;
calculate movement of the instrument according to the biopsy pattern;
move the instrument to one or more positions corresponding to the one or more sample locations.
introduce, via the fluid channel, a liquid medium at the one or more sample locations; and
transmit ultrasonic energy through the liquid medium for visualizing a target tissue site.

13. The system of claim 12, further comprising a user input device configured to receive the biopsy pattern, a command to access the biopsy pattern, or a command to calculate movement of the instrument according to the biopsy pattern.

14. The system of claim 12, further comprising at least one localization sensor in the instrument.

15. The system of claim 14, wherein the localization sensor and the ultrasound transducer element enable capture of ultrasound slice data and a position and orientation of the ultrasound transducer at each acquired slice.

16. The system of claim 14, wherein a three dimensional tissue structure model is producible from the data acquired from the ultrasound transducer element and the localization sensor.

17. The system of claim 12, further comprising a user interface screen configured to show the biopsy pattern.

18. The system of claim 12, wherein the one or more processors are configured to execute the instructions to cause the system to at least: adjust the biopsy pattern or a route representing the movement of the instrument to the one or more positions based on information received from a user.

19. The system of claim 12, further comprising a set of one or more location sensors; and wherein the one or more processors are configured to execute the instructions to cause the system to at least: calculate (1) at least one position of the set of location sensors or (2) a position of a distal end of the instrument based on a data signal from the set of one or more location sensors; and control movement to the one or more positions based on the calculated position.

20. The system of claim 12, wherein the instrument comprises:
a scope configured to reach the tissue site; and
a collection device configured to (1) be removably placed within the scope or (2) pass through the scope and collect the set of one or more biopsy samples.

21. The system of claim 12, wherein the one or more processors are further configured to execute the instructions to cause the system to at least: position the instrument to a first position, confirm receiving a first sample, and position the instrument to a second position in response to a confirmation of receiving the first sample.

22. The system of claim 12, wherein the instrument comprises a collection device configured to obtain the set of one or more biopsy samples; wherein the actuator is configured to control movements of the collection device; wherein the collection device further comprises a marker at a distal end of the collection device; and wherein the one or more processors are further configured to execute the instructions to cause the system to at least:
determine movement of the collection device according to a movement of the marker; and
adjust the one or more sample locations according to the movement of the collection device.

23. The system of claim 12, wherein the biopsy pattern comprises one or more sample positions arranged in at least two dimensions.

24. The system of claim 23, wherein the biopsy pattern comprises one or more sample positions arranged in a shape fitted to a shape of the tissue site.

25. The system of claim 23, wherein the biopsy pattern further comprises one or more penetration depths, one or more sampling velocities, one or more sampling intervals, or one or more sampling forces corresponding to the one or more sample positions.

26. The system of claim 12, wherein the one or more processors are further configured to execute the instructions to cause the system to adjust a position of a distal portion of the instrument based on, at least in part, a user input.

27. The system of claim 12, wherein the one or more processors are further configured to calculate a displacement of a location of one more samples within the target tissue site from a preprocedural model representative of the location of one or more samples within the target tissue site.

28. The system of claim 27, wherein the one or more processors are configured to execute the instructions to cause the system to at least: adjust the biopsy pattern or a route representing the movement of the instrument to the one or more positions based on, at least in part, a calculated displacement of the location of one more samples within the target tissue site from a preprocedural model representative of the location of one or more samples within the target tissue site.

29. The system of claim 27, wherein the one or more processors are further configured to adjust one or more penetration depths, one or more sampling velocities, one or more sampling intervals, or one or more sampling forces corresponding to one or more sample positions based on, at least in part, a determined displacement of the location of one more samples within the target tissue site from a preoperative model representative of the location of one or more samples within the tissue site.

30. The system of claim 27, wherein the instrument is further configured for removing, via the fluid channel, the liquid medium from one or more sample locations.

31. The system of claim 12, wherein the liquid medium comprises a perfluorochemical.

32. A robotic medical system, comprising:
an instrument driver comprising a motor and an instrument interface, the instrument interface comprising a drive element operatively coupled to the motor;
a steerable catheter comprising:
an elongate body defining a lumen,
a fluid channel extending through the elongate body to a distal end portion of the steerable catheter, the fluid channel configured for introducing a liquid medium
a control element extending through the elongate body to a distal end portion of the steerable catheter, and
an instrument base operatively coupled to the instrument interface, the instrument base comprising a pulley operatively coupled to the control element and the drive element such that actuation of the drive element by the motor actuates the pulley causing actuation of the control element and the distal end portion;
a controller having control logic configured to operate the motor of the instrument driver; and
an ultrasound transducer integrated in the steerable catheter and configured to acquire data, wherein the data acquired from the ultrasound transducer during movement through a patient is used by the controller, based on the control logic, to control the motor of the instrument driver to actuate the distal end portion of the steerable catheter to navigate the steerable catheter through the patient.

33. The robotic medical system of claim 32, further comprising at least one localization sensor in the steerable catheter.

34. The robotic medical system of claim 33, wherein the localization sensor and the ultrasound transducer enable capture of ultrasound slice data and a position and orientation of the ultrasound transducer at each acquired slice.

35. The robotic medical system of claim 34, wherein a three dimensional tissue structure model is producible from the data acquired from the ultrasound transducer and the localization sensor.

36. The robotic medical system of claim 32, further comprising a user interface screen configured to show data acquired by the ultrasound transducer.

37. The system of claim 32, wherein the controller is further configured to control the motor of the instrument driver to actuate the distal end portion of the steerable catheter based on, at least in part, a user input.

38. The robotic medical system of claim 32, wherein the controller is further configured to calculate a displacement of the location of one more anatomical features from a preprocedural model representative of the location of one or more anatomical features based on, at least in part, data acquired from the ultrasound transducer.

39. A system configured to navigate a luminal network of a patient, the system comprising:
a field generator configured to generate an electromagnetic (EM) field;
a steerable instrument comprising a set of one or more EM sensors at a distal end, a ultrasound transducer element, and a fluid channel;
a set of one or more respiration sensors;
at least one computer-readable memory having stored thereon executable instructions; and
one or more processors in communication with the at least one computer-readable memory and configured to execute the instructions to cause the system to at least:
access a preoperative model representative of the luminal network;
access a mapping between a coordinate frame of the EM field and a coordinate frame of the preoperative model;
calculate at least one position of the set of EM sensors within the EM field based on a data signal from the set of EM sensors;
calculate at least one position of one or more anatomical features based on a data signal from the ultrasound transducer element;
calculate a frequency of respiration of the patient based on a data signal from the set of one or more respiration sensors; and
determine a position of the distal end of the steerable instrument relative to the preoperative model based on the mapping, the frequency of respiration, the at least one position of one or more anatomical features, and the at least one position of the set of EM sensors within the EM field.

40. The system of claim 39, wherein the one or more processors are configured to execute the instructions to cause the system to at least:
transform one or more data signals from the set of respiration sensors into a frequency domain representation of the one or more data signals; and
identify the frequency of respiration from the frequency domain representation of the one or more data signals.

41. The system of claim 40, wherein the one or more processors are configured to execute the instructions to cause the system to at least:
apply a predictive filter to one or more data signals from the set of EM sensors, the predictive filter configured to predict respiration motion due to the respiration; and
remove components of the one or more data signals attributable to the predicted respiration motion to determine the position of the distal end of the steerable instrument relative to the preoperative model.

42. The system of claim 39, wherein the one or more processors are configured to execute the instructions to cause the system to at least calculate at least one magnitude of displacement of the set of one or more respiration sensors between an inspiration phase and an expiration phase of respiration of the patient.

43. The system of claim 42, wherein the one or more processors are configured to execute the instructions to at least:
determine at least one position of the set of EM sensors relative to the set of respiration sensors;
calculate at least one positional displacement of the set of EM sensors between the inspiration and the expiration phases based on (i) the determined at least one position of the set of EM sensors relative to the set of respiration sensors and (ii) the at least one magnitude of displacement of the set of one or more respiration sensors between the inspiration phase and the expiration phase; and
determine the position of the distal end of the steerable instrument relative to the preoperative model based on the calculated at least one positional displacement of the set of EM sensors between the inspiration phase and the expiration phase.

44. The system of claim 43, wherein the set of one or more respiration sensors comprises a first additional EM sensor positioned, in use, at a first position on a body surface and a second additional EM sensor positioned, in use, at a second position of the body surface, wherein the second position is spaced apart from the first position such that a first magnitude of displacement of the first additional EM sensor is greater than a second magnitude of displacement of the second additional EM sensor between the inspiration phase and the expiration phase.

45. The system of claim 39, wherein the one or more processors are configured to execute the instructions to cause the system to at least:
estimate a movement vector for at least a portion of the preoperative model based on the calculated at least one magnitude of displacement;
translate the preoperative model within the coordinate frame of the EM field based on the estimated movement vector; and
determine a position of the distal end of the steerable instrument based on the translated preoperative model.

46. The system of claim 39, further comprising a display, wherein the one or more processors are configured to execute the instructions to cause the system to at least:
generate a graphical representation of the position of the distal end of the steerable instrument relative to the preoperative model; and
render the generated graphical representation on the display.

47. A system for ablating tissue, the system comprising:
an elongate shaft having a movable distal portion and at least one fluid channel configured for introducing a liquid medium to a target tissue site; and
an ablation element comprising an ultrasound transducer coupled to the movable distal portion of the elongate shaft, wherein the ultrasound transducer comprises a single ultrasound transducer element having an active portion and an inactive portion,
wherein the ultrasound transducer is configured to sense a thickness of a target tissue, and
wherein the ultrasound transducer is configured to deliver a collimated beam of ultrasound energy comprising ablation energy to ablate the target tissue without contacting the target tissue with the elongate shaft or any structure disposed thereon.

48. The system of claim 47, further comprising a reflecting element operably coupled with the ultrasound transducer, the reflecting element redirecting the collimated beam of ultrasound energy emitted from the ultrasound transducer to change a direction or a pattern of the collimated beam of ultrasound energy.

49. The system of claim 48, wherein the reflecting element is configured to move relative to the ultrasound transducer so that the collimated beam of ultrasound energy is emitted at varying angles or positions.

50. The system of claim 47, further comprising a processor configured to adjust the collimated beam of ultrasound energy in response to the sensed thickness of the target tissue.

51. The system of claim 50, wherein the processor is configured to adjust one or more of frequency, a voltage, a duty cycle, a pulse length, or a position of the collimated beam of ultrasound energy in response to the sensed thickness of the target tissue.

52. The system of claim 47, further comprising a backing layer coupled to the ultrasound transducer, the backing layer configured to provide a heat sink for the ultrasound transducer.

53. The system of claim 52, wherein the backing layer comprises a plurality of grooves extending longitudinally along an outside wall of the backing layer.

54. The system of claim 47, wherein the inactive portion comprises a material configured to conduct heat away from the active portion.

55. The system of claim 47, further comprising a flow of fluid configured to cool the ultrasound transducer.

56. A method for ablating tissue, said method comprising:
providing an ablation system comprising:
an elongate shaft and an ablation element comprising at least one ultrasound transducer, wherein the ultrasound transducer comprises a single ultrasound transducer element having an active portion and an inactive portion; and
at least one fluid channel;
positioning the ablation element adjacent a target tissue site;
delivering, via at least one fluid channel of the ablation system, a liquid medium to the target tissue site to energetically couple, at least in part, at least one ultrasound transducer to a target tissue;
sensing a thickness of the target tissue with the ultrasound transducer; and
ablating at least a portion of the target tissue with a beam of ultrasound energy, thereby forming a zone of ablation comprising a continuous lesion in the target tissue.

57. The method of claim 56, wherein delivering the beam of ultrasound energy comprises reflecting the beam of ultrasound energy off of a reflecting element operably coupled with the ultrasound transducer thereby redirecting the beam of ultrasound energy and changing a direction or pattern of the beam of ultrasound energy.

58. The method of claim 57, wherein reflecting the beam of ultrasound energy comprises moving the reflecting element relative to the ultrasound transducer so that the beam of ultrasound energy is emitted at varying angles or positions.

59. The method of claim 56, further comprising directing the beam of ultrasound energy along a path such that the zone of ablation in the target tissue has a ring shape, elliptical shape, linear shape, curvilinear shape, or combinations thereof.

60. The method of claim 56, further comprising adjusting the beam of ultrasound energy in response to the sensed thickness of the target tissue.

61. The method of claim 60, wherein adjusting the beam of ultrasound energy comprises adjusting one or more of frequency, a voltage, a duty cycle, a pulse length, or a position of the beam of ultrasound energy in response to the sensed thickness of the target tissue.

62. The method of claim 56, further comprising cooling the ultrasound transducer.

63. The method of claim 56, wherein the inactive portion comprises a material configured to conduct heat away from the active portion.

64. The method of claim 56, wherein the ablation system further comprises a backing layer coupled to the ultrasound transducer, the backing layer configured to provide a heat sink for the ultrasound transducer, wherein the backing layer comprises a plurality of grooves extending longitudinally along an outside wall of the backing layer.
